(19) United States
(12) Patent Application Publication
(10) Pub. No.: US 2024/0180872 A1
(43) Pub. Date: Jun. 6, 2024

(54) METHODS AND COMPOUNDS OF CANNABIDIOL, MELATONIN AND AKBA FOR TREATING PANCREATIC CANCER

(71) Applicants: INTEGRATIVE THERAPY DISCOVERY LAB S.R.L., Camerino (IT); FONDAZIONE MARIA GUARINO AMOR ONLUS, POZZUOLI (IT)

(72) Inventors: Cristina AGUZZI, Falconara Marittima (IT); Margherita LUONGO, Pozzuoli (IT); Oliviero MARINELLI, Colonnella (IT); Massimo NABISSI, San Ginesio (IT); Laura ZEPPA, Camerino (IT)

(21) Appl. No.: 18/285,381

(22) PCT Filed: Apr. 4, 2022
(86) PCT No.: PCT/US22/23244

§ 371 Date: Oct. 2, 2023

Related U.S. Application Data
(60) Provisional application No. 63/170,449, filed on Apr. 3, 2021.
(60) Provisional application No. 63/321,649, filed on Mar. 18, 2022.

(51) Int. Cl. A61K 31/4045; A61K 31/00; A61K 31/22; A61K 45/06; A61P 35/00

(52) U.S. Cl. CPC A61K 31/4045; A61K 31/22; A61K 31/658; A61K 45/06; A61P 35/00

(57) ABSTRACT

The present disclosure relates to the use of suitable compounds such as cannabidiol (CBD), melatonin (MLT), acetyl-11-keto-beta-boswellic acid (AKBA), or combinations thereof for treatment of pancreatic cancer and/or similar conditions, diseases or disorders.

### CROSS REFERENCE TO RELATED APPLICATIONS

This application claims the benefit of priority of U.S. Provisional Patent Application No. 63/170,449 filed on Apr. 3, 2021, and U.S. Provisional Patent Application No. 63/321,649 filed Mar. 18, 2022, the contents of which are all incorporated herein by reference in their entirety.

### FIELD OF THE INVENTION

The present disclosure relates to the use of a multi-compound composition including Cannabidiol (CBD), Melatonin (MLT) and AKBA (acetyl-11-keto-beta-boswellic acid) for treatment of pancreatic neoplasms, and for treatment of pancreatic cancer. Antitumor efficacy of cannabidiol and melatonin, in a nude mice orthotopic pancreatic tumor model was tested.

### BACKGROUND OF THE INVENTION

Pancreatic cancer (PC) is a lethal malignancy with a 5-year survival of approximately 5-9% [1,2]. The most common and aggressive type, among pancreatic malignancies, is pancreatic ductal adenocarcinoma (PDAC), an infiltrating neoplasm with glandular differentiation, that is the fourth cause of cancer related death worldwide [2-4]. The development of PC is related to environmental and lifestyle risks, but also pathological conditions linked to chronical inflammations and, for a subgroup of pancreatic cancer (PC) patients, germline mutations in Breast Cancer Type 1/2 (BRCA1/2), ATM Serine/Threonine Kinase (ATM), MutL Homolog 1 gene (MLH1), TP53 or Cyclin Dependent Kinase Inhibitor 2A (CDKN2A), which are considered further risk factors [5,6]. Indeed, somatic mutations as in oncogene (KRAS) and onco-suppressor genes (TP53, CDKN2A, SMAD Family Member 4 SMAD4), that support cancer aggressiveness, are important for the diagnosis of this malignant phenotype [2,7].

CBD has been demonstrated to interact with different receptors, such as cannabinoid receptors (CB1, CB2), G Protein-Coupled Receptor 55 (GPR55), transient potential receptors (TRPV1, TRPV2, TRPV3, TRPV4, TRPM8, TRPA1), and peroxisome proliferator-activated receptor (PPAR-γ). CBD can also act in an unknown independent-receptors manner [3-7]. Moreover, CBD was found able to influence significant changes in the expression profile of genes strongly involved in PC leading to the inhibition of cell viability, invasion, increasing cell death and acts synergically with chemodrugs (Gemcitabine; GEM or Paclitaxel; PTX) used in PC therapies (8). In vivo study demonstrated that a combination of Cannabidiol (CBD) with Tetrahydrocannabinol (THC) or Gemcitabine (GEM) in murine model of PDAC, increased a survival nearly three times longer, compared to mice treated with vehicle or GEM alone [9, 10].

MLT induces apoptotic cell death in the human pancreatic carcinoma cell line MIAPaCa-2 via the suppression of NF-κB and activation of extracellular signal-regulated kinase (ERK) and JNK. Some reports suggest that ERK pathway activation affects a survival signal that weakens proapoptotic effects via activating JNK. The MAPK pathway plays an important role in cell survival and proliferation.

AKBA is a derivative of Boswellia acid, which is the main component of a gum resin from Boswellia serrata. AKBA has been used traditionally to treat a number of inflammatory diseases, including osteoarthritis, chronic colitis, ulcerative colitis, Crohn's disease, and bronchial asthma, but its mechanism of action is poorly understood. It was reported that boswellic acid directly interacts with IκB kinases (12) and suppresses NF-κB-regulated gene expression (13). In addition, boswellic acid has been shown to potentiate apoptosis in several types of tumor cells, including colon cancer (14), prostate cancer (15), fibrosarcoma (16), hepatoma (17), and malignant glioma (18) cells, through caspase-8 activation (14) and death receptor 5-mediated signaling (15). In another report, AKBA was investigated in the inhibition of invasion of pancreatic and breast cancer cells (19).

In the art, WO2011005310A1 generally discloses the use of CBD and MLT for preparing a medicinal product for the treatment of cancer, but the experiments disclosed in the application only tested these compounds at low doses. WO2020081513A1, combined more than a hundred compounds. However, this publication separates compounds in three groups, and in the claims (page 87) they declare that the patent is related to a combination of one compound from group one with one from group two and one from group three. Cannabidiol, melatonin, and Boswellia extract are in the same group (three), and were not combined or tested together.

PC is still considered incurable and the discovery of new treatments to improve the currently available therapies remains desirable. Thus, it is desirable to focus on the treatment of pancreatic cancer.

### SUMMARY OF THE INVENTION

It is an object of the present invention to improve upon the deficiencies of the cited art and provide a treatment of pancreatic cancer.

Specifically, it is an object of the present invention to provide a multi-component composition of CBD-MLT-AKBA for the treatment of PC.

In experimentation, it was unexpectedly found that CBD, alone, and in combinations with MLT and AKBA, induced cytotoxicity in PC cell lines. A dose- dependent effect in all PC cell lines was observed for all individual compound with the efficacy in reducing cell viability. The combination of CBD, MLT and AKBA was observed to have the highest efficacy in reducing cell viability followed by combinations comprising CBD and one of the other compounds.

To evaluate the cytotoxic mechanism, PC cell lines were treated daily with different cytotoxic doses of CBD, MLT and AKBA, alone and in combination, and the results evidenced that all the compositions induced cell death.

Moreover, CBD, MLT and AKBA were demonstrated to act synergically and to influence different cancer pathways involved in PC progression. Accordingly, the present disclosure includes a method of treating PC in a subject in need thereof, comprising administering an effective amount of at least one of the compounds.

In certain embodiments, the at least one compound comprises CBD, MLT, AKBA or combinations thereof. In another embodiment of the present disclosure, the at least one compound is CBD, MLT, AKBA or combinations thereof.

In certain embodiments, the at least one compound is CBD.

In certain embodiments, the at least one compound is MLT.

In certain embodiments, the at least one compound is AKBA.

In certain embodiments, the at least one compound is a combination of CBD and MLT.

In certain embodiments, the at least one compound is a combination of CBD and AKBA.

In certain embodiments, the at least one compound is a combination of MLT and AKBA.

In certain embodiments, the at least one compound is a combination of CBD, MLT and AKBA.

In certain embodiments, the neoplasm is PC.

In certain embodiments, the at least one compound is administered or for use in combination with at least one other anticancer treatment.

In certain embodiments, the subject is a human.

In certain embodiments, the CBD comprises conjugated CBD formulation, synthetic CBD and soluble CBD.

In certain embodiments, the at least one compound includes Boswellia extract.

In certain embodiments, the at least one compound includes AKBA.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments but should rather be given the broadest interpretation consistent with the description as a whole.

### BRIEF DESCRIPTION OF THE FIGS.

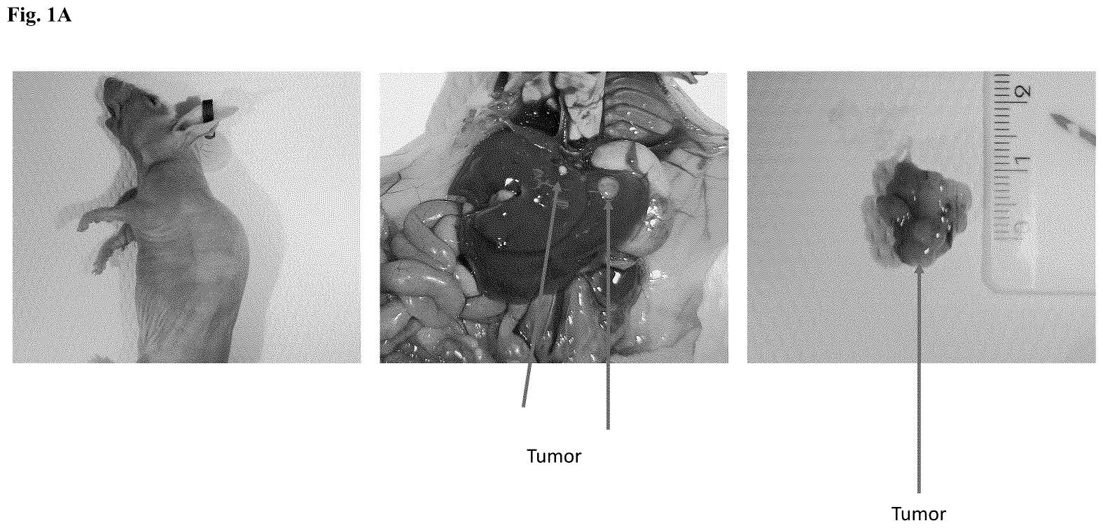

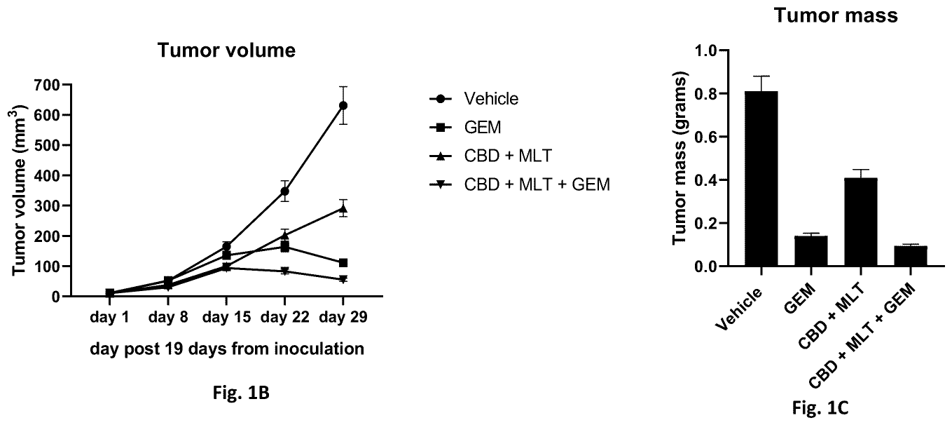

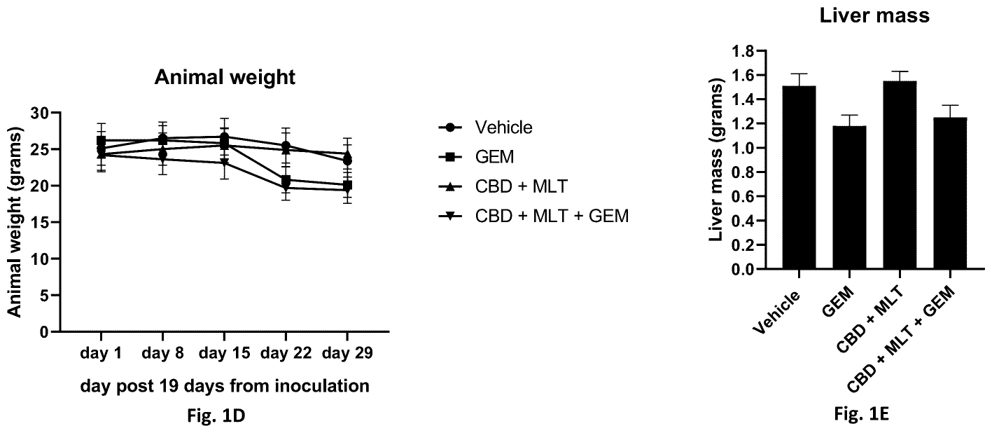

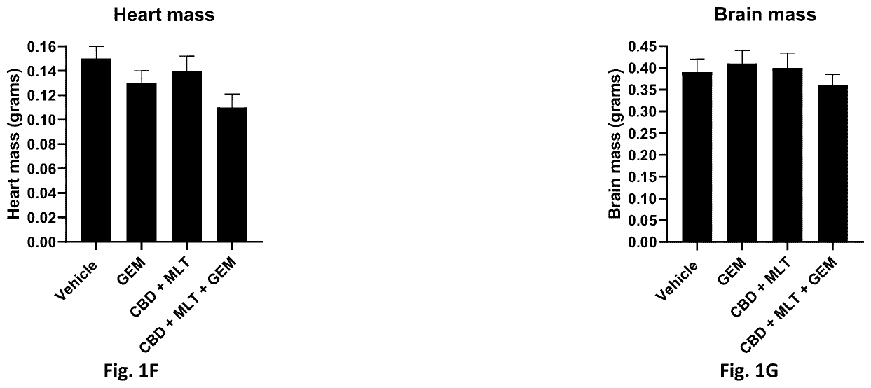

FIGS. 1A-1G. Antitumor activity of Cannabidiol (CBD), melatonin (MLT) plus gemcitabine (GEM) in vivo in an Athymic Nude-mice pancreatic tumor orthotopic xenograft mouse model.

FIG. 1A—Representative mice injected orthotopically with PANC-1 cells and xenograph tumor.

FIG. 1B—Tumor volume progression timeline with experimental treatment time points.

FIG. 1C—Tumor mass at timepoint.

FIG. 1D—Animal weight at timepont.

FIGS. 1E-1G) Liver, Heart and Brain weight weighed at 30 days to estimate tissue toxicity. These animal experiments were repeated once (n=5 mice per treatment group). Data are presented as mean±S.D. *p<0.05 versus the corresponding control groups. #p<0.05 versus GEM.

### DETAILED DESCRIPTION OF THE INVENTION

The application hereby incorporates the contents of Appendices 1-6 into this application in their entirety.

### I. Definitions

Unless otherwise indicated, the definitions and embodiments described in this and other sections are intended to be applicable to all embodiments and aspects of the disclosure herein described for which they would be understood to be suitable by a person skilled in the art.

As used herein, the words “comprising” (and any form thereof, such as “comprise” and “comprises”), “having” (and any form thereof, such as “have” and “has”), “including” (and any form thereof, such as “include” and “includes”) or “containing” (and any form thereof, such as “contain” and “contains”), are inclusive or open-ended and do not exclude additional, unrecited elements or process/method steps. As used herein, the word “consisting” and its derivatives are intended to be close-ended terms that specify the presence of the stated features, elements, components, groups, integers and/or steps, and also exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of these features, elements, components, groups, integers and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the term it modifies.

As used in this disclosure, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is present or used.

The term “cannabidiol” and the abbreviation “CBD” as used herein refer to the phytocannabinoid 2-[(1R,6R)-3-methyl-6-prop-1-en-2-ylcyclohex-2-en-1-yl]-5-pentylbenzene-1,3-diol of the structure:

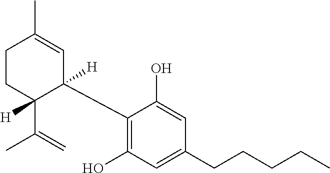 [SMILES: C=C(C)[C@@H]1CCC(C)=C[C@H]1c1c(O)cc(CCCCC)cc1O]

The term “Melatonin” and the abbreviation “MLT” as used herein refer to N-[2-(5-methoxy-1H-indol-3-yl)ethyl]acetamide of the structure:

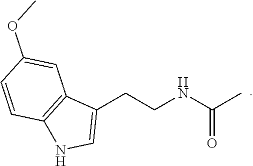 [SMILES: COc1ccc2[nH]cc(CCNC(C)=O)c2c1]

The term “AKBA” as used herein refer to the (3R,4R,4aR,6aR,6bS,8aR,11R,12S,12aR,14aR,14bS)-3-acetyloxy-4,6a,6b,8a,11,12,14b-heptamethyl-14-oxo-1,2,3,4a,5,6,7,8,9,10,11,12,12a, 14a-tetradecahydropicene-4-carboxylic acid of the structure:

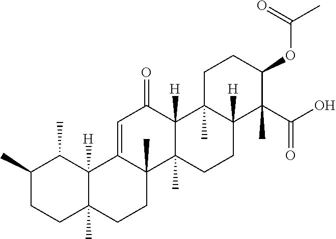

The term “subject” as used herein includes all members of the animal kingdom including mammals. In an embodiment, the subject is a human.

The term “pharmaceutically acceptable” means compatible with the treatment of subjects, for example, mammals such as humans.

The term “enteral” as used herein means taken into the body or administered or used in a manner that is through the gastrointestinal tract.

The term “parenteral” as used herein means taken into the body or administered or used in a manner other than through the gastrointestinal tract.

The terms “to treat”, “treating”, “treatment” and the like as used herein and as is well understood in the art, refer to an approach for obtaining beneficial or desired results, including clinical results. Beneficial or desired results include, but are not limited to, alleviation or amelioration of one or more symptoms of a disease, condition or disorder such as pancreatic cancer, diminishment of the extent of the disease, condition or disorder such as pancreatic cancer, stabilized (i.e. not worsening) disease, condition or disorder such as pancreatic cancer, delay or slowing of the progression of the disease, condition or disorder such as pancreatic cancer, amelioration or palliation of the state of the disease, condition or disorder such as PC and/or remission (whether partial or total) of the disease, condition or disorder such as pancreatic cancer, whether detectable or undetectable. “To treat”, “treating”, “treatment” and the like as used herein also include prophylactic treatment of the disease, condition or disorder such as PC. For example, a subject with early stage PC is treated to prevent or delay progression or alternatively a subject in remission is treated to prevent or delay recurrence.

As used herein, the term “effective amount” and the like as used herein means an amount effective, at dosages and for periods of time necessary to achieve a desired result. For example, in the context of treating PC, an effective amount of the at least one compound is an amount that, for example, reduces the PC compared to the PC without administration of the at least one compound.

By “reducing the PC” it is meant, for example, reducing the number of PC cells, reducing the symptoms of the PC and/or slowing the advancement of the PC. Effective amounts may vary according to factors such as the disease state, age, sex and/or weight of the subject. The amount of the at least one compound that will correspond to such an amount will vary depending upon various factors, such as the given compound or combination thereof, the pharmaceutical formulation, the route of administration or use, the identity of the subject being treated, and the like, but can nevertheless be routinely determined by one skilled in the art having reference to this disclosure.

### II. Methods and Uses

The cannabinoid CBD, MLT, and AKBA alone, and in combinations, induced cytotoxicity in PDAC cell lines. To evaluate the cytotoxic mechanism, PDAC cell lines were treated daily with different doses for each compound and the results evidenced that all cell death and modulate different pathways involved in PDAC. This was confirmed via cytotoxicity assay and Gene expression analysis.

Accordingly, the present disclosure includes a method of treating a pancreatic neoplasm in a subject in need thereof, comprising administering an effective amount of at least one compound to the subject. The present disclosure also includes a use of an effective amount of at least one compound for treatment of pancreatic neoplasm in a subject in need thereof, with the proviso that the at least one compound comprises CBD. The present disclosure also includes a use of an effective amount of at least one compound for preparation of a medicament for treatment of a pancreatic neoplasm in a subject in need thereof, with the proviso that the at least one compound comprises MLT. The present disclosure also includes at least one compound for use to treat a pancreatic neoplasm in a subject in need thereof, with the proviso that the at least one compound comprises AKBA.

In certain embodiments, the at least one compound comprises, consists essentially of or consists of (or “is”) CBD, MLT, AKBA or combinations thereof.

However, a dose dependent effect in all PDAC cell lines was also observed for the individual compounds CBD, MLT, AKBA tested in the present examples. Accordingly, in certain embodiments, the at least one compound consists essentially of or consists of (or “is”) CBD. In another embodiment, the at least one compound consists essentially of MLT. In a further embodiment, the at least one compound consists of (or “is”) AKBA.

The combination of CBD with MLT was surprisingly observed to have the highest efficacy in reducing cell viability followed by combinations comprising AKBA. Several of the particular doses for these combinations were observed to be more effective compared to the sum of the individual compounds.

Accordingly, in certain embodiments, the at least one compound consists essentially of or consists of (or “is”) a combination of CBD and MLT. In another embodiment, the at least one compound consists essentially of a combination of CBD and AKBA. In a further embodiment, the at least one compound at least consists of (or “is”) a combination of MLT and AKBA.

In an embodiment, the combination of at least one compound that is CBD, MLT is a combination comprising AKBA. In another embodiment of the present disclosure, the combination of at least one compound that is MLT or AKBA is a combination comprising CBD.

In an embodiment, the at least one compound consists essentially of or consists of (or “is”) a combination of CBD, MLT and AKBA.

In an embodiment, the at least one compound is an individual compound. In another embodiment, the at least one compound is a combination of two compounds. In a further embodiment, the at least one compound is a combination of three compound.

In an embodiment, the cancer is PC.

In an embodiment, the at least one compound is administered or for use in combination with at least one other anticancer treatment.

In an embodiment, the subject is a human.

The at least one compound is administered to a subject or used in a variety of forms depending on the selected route of administration or use, as will be understood by those skilled in the art. In an embodiment, the at least one compound is administered to the subject or used, for example, by enteral or parenteral routes, and the at least one compound formulated accordingly.

Enteral administration or use includes all suitable routes involving the gastrointestinal tract, for example, oral, buccal, sublingual, nasal and rectal. In an embodiment of the present disclosure, the enteral administration or use of the at least one compound is oral administration or use; i.e. the at least one compound is administered orally or is for oral use, as the as the case may be. Formulations suitable for oral administration or use may be prepared by methods known to a person skilled in the art.

Parenteral administration or use includes intravesical, intravenous, intraperitoneal, subcutaneous, intramuscular, transepithelial, intrapulmonary, intrathecal, and topical modes of administration or use. Formulations suitable for parenteral administration or use may be prepared by known methods by a person skilled in the art.

Treatment methods comprise administering to a subject or use of an effective amount of the at least one compound and optionally consist of a single administration or use, or alternatively comprise a series of administrations or uses. For example, the at least one compound is administered or used at least once a week. However, in another embodiment, the at least one compound is administered to the subject or used from one time per three weeks or one time per week to once daily for a given treatment or use. In another embodiment, the at least one compound is administered or used 2, 3, 4, 5 or 6 times daily. The length of the treatment period depends on a variety of factors, such as the severity of the disease, disorder or condition such as PC (e.g. disease stage), the age and/or sex of the subject, and the activity and/or formulation of the at least one compound and/or a combination thereof. It will also be appreciated that the effective amount of the at least one compound used for the treatment or use may increase or decrease over the course of a particular treatment regime or use. Changes in dosage may result and become apparent by standard diagnostic assays known in the art. In some embodiments of the present disclosure, chronic administration or use may be required. For example, the at least one compound is administered or used in an amount and for a duration sufficient to treat the subject.

In embodiments, wherein the at least one compound is a combination of at least two compounds, the compounds are either used or administered separately in time and/or in mode of administration or use (i.e. different routes of administration or use) or they are administered or for use together in the same pharmaceutical preparation and/or at the same time, which may depend, for example, on the identity of the compounds.

In an embodiment, the at least two compounds are used or administered separately in time and/or in mode of administration or use.

In another embodiment, the at least two compounds are administered or for use contemporaneously. As used herein, contemporaneous administration or use, for example, of two substances to a subject means providing the first compound and the second compound, so that the pharmacological effects of the first compound and the second compound are present in the subject at the same time. The exact details of the administration or use will depend on the pharmacokinetics of the first compound and the second compound in the presence of each other and can include administering or use of the first compound and the second compound within a few hours of each other, or even administering or use of the first compound and the second compound within 24 hours, or 48 hours or greater of administration or use of the other, if the pharmacokinetics are suitable. Design of suitable dosing regimens is routine for one skilled in the art. In some embodiments, the at least two compounds are administered or used substantially simultaneously, i.e. within minutes of each other or in a single composition that comprises both substances. In another embodiment, the at least two compounds are administered to a subject or for use in a non-contemporaneous fashion. In a further embodiment, the at least two compounds are administered to a subject or for use in a contemporaneous fashion followed by, or alternating with, administration or use in a non-contemporaneous fashion.

The dosage of the at least one compound can vary depending on many factors such as the pharmacodynamic properties of the compound or combination thereof, the mode of administration or use, the age, health and weight of the subject, the nature and extent of the symptoms of the disease, disorder or condition such as pancreatic cancer, the frequency of the treatment or use and the type of concurrent treatment or use, if any, and/or the clearance rate of the compound in the subject. One of skill in the art can determine the appropriate dosage having regard to the above factors. In an embodiment, the at least one compound is administered or used initially in a suitable dosage that is optionally adjusted as desired, depending on the clinical response. As a representative example, oral dosages of the at least one compound may range from less than 1 mg per day to 10 g per day for a human subject. In an embodiment of the present disclosure, the at least one compound is formulated in a pharmaceutical composition suitable for oral administration or use and the compounds are, for example, present in an amount of about 0.001, 0.01, 0.1, 0.25, 0.5, 0.75, 1.0, 5.0, 7.5, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950, 1000 up to 10.000 mg of active ingredient per dose. In another embodiment of the present disclosure, the at least one compound is administered or used in a single daily dose or the total daily dose may be divided into two, three or four daily doses.

The following non-limiting examples are illustrative of the present disclosure:

### EXAMPLES

### Example 1: Effect of CBD, MLT, AKBA and Their Combinations Thereof on PDAC Cell Lines

This study evaluated the effects of three compounds named: Cannabidiol (CBD), Melatonin (MLT) and acetyl-11-keto-beta-boswellic acid (AKBA) alone and in combination, in regulating cell survival in human pancreatic ductal adenocarcinoma (PDAC) cell lines.

### Materials and Methods

### Cell Lines

Human pancreatic ductal adenocarcinoma (PANC-1 and MIAPaCa-2) cell lines were purchased by Sigma Aldrich (Milan, Italy) and cultured in DMEM high glucose medium (EuroClone, Milan, Italy) supplemented with 10% of fetal bovine serum (FBS), 2 mM L-glutamine, 100 IU/mL penicillin, 100 mg streptomycin and 1 mM sodium pyruvate. Cell lines were maintained at 37° C. with 5% CO2 and 95% of humidity. The glioblastoma U251 cell lines (grade IV), obtained from European Collection of Cell Cultures (ECACC, Salisbury, UK), were maintained in Dulbecco's modified Eagle's medium (DMEM, Lonza Bioresearch, Basel, Switzerland) supplemented with 10% heat inactivated fetal bovine serum (FBS), 2 mmol/L L-glutamine, 100 IU/mL penicillin, 100 μg streptomicin at 37° C., 5% CO2, and 95% humidity. Primary endometrial cancer cell line PCEM004a cells were grown in McCoy's Medium (Lonza, Milan, Italy), supplemented with 10% FBS, 100 IU/mL penicillin, 100 mg streptomycin, while all the primary cell lines were grown in RPMI1640, supplemented with 20% FBS, 2 mM/L glutamine, 100 IU/mL penicillin, 100 mg streptomycin.

### Cytotoxicity Assay

3×10⁴ cells/mL were seeded in 96-well plates in a final volume of 100 L/well. After one day of incubation, compounds or vehicles, alone or in combination, were added and six replicates were used for each treatment and all experiments were repeated three times. In all the experiments, the treatment was daily added, after washing with fresh medium. After 72 hours cell viability was investigated by adding 0.8 mg/mL of 3-[4,5-dimethylthiazol-2-yl]-2,5 diphenyl tetrazolium bromide (MTT) (Sigma Aldrich) to the media. After 3 h the supernatant was removed, and the pellet of salt crystals was solubilized with 100 μL/well of DMSO. The absorbance of the sample against a background control was measured at 570 nm using an ELISA reader microliter plate (BioTek Instruments, Winooski, VT, USA).

### RNA Isolation, Reverse Transcription and Quantitative Real-Time PCR and TaqMan Array

Total RNA from untreated or CBD, MLT, AKBA alone and in combination treated cell lines was extracted using Rneasy Mini kit (Qiagen, Milan, Italy). One microgram of total RNA from each sample was subjected to reverse transcription in a total volume of 20 μL using the High-Capacity cDNA Archive Kit (Applied Biosystem, Foster City, PA, USA) according to the instructions. cDNAs were analyzed by qRT-PCR performed using an IQ5 Multicolor Real time PCR Detection system. Quantitative real-time gene expression was performed with the TaqMan® Array Human Pancreatic Adenocarcinoma 96-well Plate, containing 92 assays to pancreatic adenocarcinoma associated genes and 4 assays to candidate endogenous control genes, was purchased (Thermo Fisher, Grand Island, NY, USA) and used to evaluate the treatments in modulating PDAC-related genes (Table 1). Measurement of different housekeeping genes (GAPDH; HPRT1; GUSB) on the samples was used to normalize mRNA content. The gene expression levels of treated cell lines were expressed as relative fold compared with untreated or vehicle-treated cells.

### Statistical Analysis

The data presented represent the mean with standard deviation (SD) of at least 3 independent experiments. Synergistic activity of the CBD and chemotherapeutic drugs combination was calculated by the Chou-Talalay method, which provides the theoretical basis for the combination index (CI)-isobologram equation. This method allows quantitative determination of drug interactions, where CI<1,=1, and >1 indicates synergism, additive and antagonism, respectively. Based on these algorithms, computer software, CompuSyn 3.0.1 version (CompuSyn Software, ComboSyn, Inc., Paramus, NJ, USA, 2007) was used for automatically determining synergism and antagonism at all doses or effect levels. The statistical significance was determined by Student's t-test and by One Way-Anova and TwoWay-Anova with Bonferroni's post-test; *p<0.05.

TABLE 1
TaqMan Array, Human Pancreatic Adenocarcinoma gene list.

| # | Assay ID | Gene Symbol |
|---|---|---|
| 1 | Hs03003631_g1 | 18S |
| 2 | Hs99999905_m1 | GAPDH |
| 3 | Hs99999909_m1 | HPRT1 |
| 4 | Hs99999908_m1 | GUSB |
| 5 | Hs00178289_m1 | AKT1 |
| 6 | Hs01086102_m1 | AKT2 |
| 7 | Hs00178533_m1 | AKT3 |
| 8 | Hs00388776_m1 | ARHGEF7 |
| 9 | Hs99999018_m1 | BCL2 |
| 10 | Hs00236329_m1 | BCL2L1 |
| 11 | Hs00153353_m1 | BIRC5 |
| 12 | Hs00269944_m1 | BRAF |
| 13 | Hs01037414_m1 | BRCA2 |
| 14 | Hs00153138_m1 | CCNA2 |
| 15 | Hs99999188_m1 | CCNB1 |
| 16 | Hs00765553_m1 | CCND1 |
| 17 | Hs00153380_m1 | CCND2 |
| 18 | Hs01026536_m1 | CCNE1 |
| 19 | Hs00372959_m1 | CCNE2 |
| 20 | Hs00741586_mH | CDC42 |
| 21 | Hs01548894_m1 | CDK2 |
| 22 | Hs00175935_m1 | CDK4 |
| 23 | Hs00355782_m1 | CDKN1A |
| 24 | Hs00153277_m1 | CDKN1B |
| 25 | Hs00923894_m1 | CDKN2A |
| 26 | Hs00365249_m1 | CDKN2B |
| 27 | Hs00176227_m1 | CDKN2C |
| 28 | Hs00176481_m1 | CDKN2D |
| 29 | Hs00559367_m1 | CYP2E1 |
| 30 | Hs00153451_m1 | E2F1 |
| 31 | Hs00605457_m1 | E2F3 |
| 32 | Hs00608098_m1 | E2F4 |
| 33 | Hs01099999_m1 | EGF |
| 34 | Hs01076078_m1 | EGFR |
| 35 | Hs00428286_g1 | ELK1 |
| 36 | Hs01001580_m1 | ERBB2 |
| 37 | Hs01128659_m1 | FIGF |
| 38 | Hs00157817_m1 | GRB2 |
| 39 | Hs00181813_m1 | HBEGF |
| 40 | Hs00743767_sH | HSP90AA1 |
| 41 | Hs01547656_m1 | IGF1 |
| 42 | Hs00985639_m1 | IL6 |
| 43 | Hs01026983_m1 | JAK1 |
| 44 | Hs01078136_m1 | JAK2 |
| 45 | Hs00169663_m1 | JAK3 |
| 46 | Hs00911700_m1 | KDR |
| 47 | Hs00174029_m1 | KIT |
| 48 | Hs00364282_m1 | KRAS |
| 49 | Hs00605615_mH | MAP2K1 |
| 50 | Hs00360961_m1 | MAP2K2 |
| 51 | Hs01046830_m1 | MAPK1 |
| 52 | Hs00385075_m1 | MAPK3 |
| 53 | Hs99999008_m1 | MDM2 |
| 54 | Hs00899658_m1 | MMP1 |
| 55 | Hs01548727_m1 | MMP2 |
| 56 | Hs00968308_m1 | MMP3 |
| 57 | Hs01042796_m1 | MMP7 |
| 58 | Hs00234579_m1 | MMP9 |
| 59 | Hs00765730_m1 | NFKB1 |
| 60 | Hs00174517_m1 | NFKB2 |
| 61 | Hs01062011_m1 | NOTCH1 |
| 62 | Hs00180679_m1 | PIK3CA |
| 63 | Hs00927728_m1 | PIK3CB |
| 64 | Hs00192399_m1 | PIK3CD |
| 65 | Hs00381459_m1 | PIK3R1 |
| 66 | Hs00178181_m1 | PIK3R2 |
| 67 | Hs00153133_m1 | PTGS2 |
| 68 | Hs01025984_m1 | RAC1 |
| 69 | Hs01032884_m1 | RAC2 |
| 70 | Hs00234119_m1 | RAF1 |
| 71 | Hs01078066_m1 | RB1 |
| 72 | Hs00968436_m1 | REL |
| 73 | Hs00153294_m1 | RELA |
| 74 | Hs00232399_m1 | RELB |
| 75 | Hs00357608_m1 | RHOA |
| 76 | Hs00269660_s1 | RHOB |
| 77 | Hs00183425_m1 | SMAD2 |
| 78 | Hs00969210_m1 | SMAD3 |
| 79 | Hs00929647_m1 | SMAD4 |
| 80 | Hs00362308_m1 | SOS1 |
| 81 | Hs00178494_m1 | SRC |
| 82 | Hs01013989_m1 | STAT1 |
| 83 | Hs01013123_m1 | STAT2 |
| 84 | Hs00374280_m1 | STAT3 |
| 85 | Hs00273500_m1 | STAT5B |
| 86 | Hs00598625_m1 | STAT6 |
| 87 | Hs00608187_m1 | TGFA |
| 88 | Hs00998133_m1 | TGFB1 |
| 89 | Hs00234244_m1 | TGFB2 |
| 90 | Hs01086000_m1 | TGFB3 |
| 91 | Hs00610318_m1 | TGFBR1 |
| 92 | Hs00234253_m1 | TGFBR2 |
| 93 | Hs01034249_m1 | TP53 |
| 94 | Hs00900055_m1 | VEGFA |
| 95 | Hs00173634_m1 | VEGFB |
| 96 | Hs01099203_m1 | VEGFC |

### II. Results

Cytotoxicity in PDAC cell lines: The effect of CBD, MLT and AKBA in reducing cell viability was evaluated at 72 hours, post daily administration, in PDAC cell lines. Cells were treated with different doses of CBD, MLT and AKBA (up to 1 mg) and percentage of cell viability was evaluated by the MTT assay. The results showed a dose dependent effect in all PDAC cell lines for all of the compounds (Table 2). To evaluate a potential synergism between the CBD, MLT and AKBA, a MTT assay combining CBD, MLT and AKBA at lower cytotoxic doses in pairs and triplets (Table 2) were performed. The compounds were administered daily for 72 hours, as in previous experiments at the desired doses. Each combination was evaluated in six wells and in three separate experiments. MTT assay was used to analyse the cell cytotoxicity. The values in the tables are represented as % of cell viability compared to vehicle-treated cells. The standard deviation of the data reported was ≤10%. Regarding the results obtained with the combinations, some combinations were more effective than the single and pairs, with high efficacy that was obtained with the triple combinations.

TABLE 2
Cytotoxic effects of CBD, MLT and AKBA alone and in
combination in PANC-1 and MIAPaCa-2 cell lines.

| | PANC-1 | MIAPaCa-2 |
|---|---|---|
| | CELL LINE | CELL LINE |
| | (% VIABILITY ± | (% VIABILITY ± |
| | S.D.) | S.D.) |
| COMPOUND (micrograms/ml) | | |
| AKBA 5 | 92.1 ± 5.3 | 110.2 ± 7.3 |
| AKBA 7.5 | 86.2 ± 6.6 | 74.8 ± 6.2 |
| AKBA 10 | 78.2 ± 5.9 | 55.3 ± 4.8 |
| AKBA 15 | 55.3 ± 4.4 | 12.1 ± 0.9 |
| CBD 1.9 | 86.5 ± 7.1 | 74.8 ± 7.2 |
| CBD 3.8 | 66.4 ± 5.5 | 29.5 ± 1.9 |
| CBD 7.6 | 13.1 ± 1.1 | 9.2 ± 0.4 |
| CBD 15.2 | 7.9 ± 0.3 | 5.2 ± 0.4 |
| MLT 100 | 100.4 ± 5.7 | 100.2 ± 8.8 |
| MLT 200 | 97.4 ± 6.1 | 87.8 ± 8.2 |
| MLT 400 | 83.9 ± 6.1 | 78.2 ± 8.5 |
| MLT 800 | 19.2 ± 1.1 | 12.5 ± 0.8 |
| AKBA 5 + CBD 1.9 | 76.4 ± 4.5 | 65.7 ± 5.3 |
| AKBA 7.5 + CBD 1.9 | 77.6 ± 6.1 | 46.9 ± 3.8 |
| AKBA 10 + CBD 1.9 | 74.2 ± 6.1 | 48.6 ± 3.9 |
| AKBA 15 + CBD 1.9 | 43.2 ± 3.0 | 3.4 ± 0.1 |
| AKBA 5 + CBD 3.8 | 60.4 ± 5.5 | 30.5 ± 2.8 |
| AKBA 7.5 + CBD 3.8 | 51.7 ± 5.0 | 19.7 ± 1.1 |
| AKBA 10 + CBD 3.8 | 56.5 ± 4.4 | 26.1 ± 1.3 |
| AKBA 15 + CBD 3.8 | 46.6 ± 3.3 | 6.4 ± 5.1 |
| AKBA 5 + CBD 7.6 * | 8.5 ± 0.4 | 6.7 ± 0.4 |
| AKBA 7.5 + CBD 7.6 * | 6.1 ± 0.5 | 5.8 ± 0.1 |
| AKBA 10 + CBD 7.6 * | 7.5 ± 0.2 | 4.2 ± 0.2 |
| AKBA 15 + CBD 7.6 * | 5.1 ± 0.3 | 3.6 ± 0.1 |
| AKBA 5 + CBD 15.2 * | 3.4 ± 0.1 | 2.2 ± 0.1 |
| AKBA 7.5 + CBD 15.2 * | 0 | 0 |
| AKBA 10 + CBD 15.2 * | 0 | 0 |
| AKBA 15 + CBD 15.2 * | 0 | 0 |
| AKBA 5 + MLT 100 | 73.6 ± 4.4 | 69.4 ± 3.9 |
| AKBA 7.5 + MLT 100 | 67.1 ± 5.9 | 59.2 ± 5.1 |
| AKBA 10 + MLT 100 | 68.5 ± 4.9 | 41.5 ± 2.7 |
| AKBA 15 + MLT 100 * | 38.9 ± 3.1 | 26.5 ± 1.9 |
| AKBA 5 + MLT 200 | 65.4 ± 5.5 | 89.4 ± 8.1 |
| AKBA 7.5 + MLT 200 | 60.8 ± 5.9 | 87.4 ± 6.6 |
| AKBA 10 + MLT 200 | 66.5 ± 6.1 | 42.5 ± 4.1 |
| AKBA 15 + MLT 200 * | 38.9 ± 3.2 | 35.4 ± 2.6 |
| AKBA 5 + MLT 400 | 58.9 ± 5.1 | 63.5 ± 5.8 |
| AKBA 7.5 + MLT 400 | 70.3 ± 6.6 | 68.8 ± 6.2 |
| AKBA 10 + MLT 400 * | 59.5 ± 4.2 | 49.2 ± 3.9 |
| AKBA 15 + MLT 400 * | 42.6 ± 4.1 | 9.7 ± 0.1 |
| AKBA 5 + MLT 800 * | 12.2 ± 0.4 | 7.5 ± 0.3 |
| AKBA 7.5 + MLT 800 * | 0 | 0 |
| AKBA 10 + MLT 800 * | 0 | 0 |
| AKBA 15 + MLT 800 * | 0 | 0 |
| CBD 1.9 + MLT 100 | 83.0 ± 6.5 | 61.4 ± 4.6 |
| CBD 1.9 + MLT 200 | 71.0 ± 5.9 | 53.3 ± 5.1 |
| CBD 1.9 + MLT 400 | 66.4 ± 6.1 | 80.2 ± 7.2 |
| CBD 1.9 + MLT 800 * | 0 | 0 |
| CBD 3.8 + MLT 100 | 47.4 ± 4.1 | 16.5 ± 0.9 |
| CBD 3.8 + MLT 200 * | 43.8 ± 3.3 | 20.5 ± 1.7 |
| CBD 3.8 + MLT 400 * | 32.2 ± 2.6 | 27.4 ± 1.9 |
| CBD 3.8 + MLT 800 * | 0 | 0 |
| CBD 7.6 + MLT 100 * | 8.5 ± 0.5 | 4.6 ± 0.1 |
| CBD 7.6 + MLT 200 * | 0 | 0 |
| CBD 7.6 + MLT 400 * | 0 | 0 |
| CBD 7.6 + MLT 800 * | 0 | 0 |
| CBD 15.2 + MLT 100 * | 0 | 0 |
| CBD 15.2 + MLT 200 * | 0 | 0 |
| CBD 15.2 + MLT 400 * | 0 | 0 |
| CBD 15.2 + MLT 800 * | 0 | 0 |
| FOR TRIPLICATE COMPOSITIONS WE USED | | |
| ONLY COMBINATION OF MOST EFFECTIVE | | |
| DOSES | | |
| AKBA 7.5 + CBD 3.8 + MLT 200 * | 59.5 ± 5.5. | 15.9 ± 1.2 |
| AKBA 7.5 + CBD 3.8 + MLT 400 * | 0 | 0 |
| AKBA 7.5 + CBD 3.8 + MLT 800 * | 0 | 0 |
| AKBA 7.5 + CBD 7.6 + MLT 200 * | 0 | 0 |
| AKBA 7.5 + CBD 7.6 + MLT 400 * | 0 | 0 |
| AKBA 7.5 + CBD 7.6 + MLT 800 * | 0 | 0 |
| AKBA 7.5 + CBD 15.2 + MLT 200 * | 0 | 0 |
| AKBA 7.5 + CBD 15.2 + MLT 400 * | 0 | 0 |
| AKBA 7.5 + CBD 15.2 + MLT 800 * | 0 | 0 |
| AKBA 10 + CBD 3.8 + MLT 200 * | 0 | 0 |
| AKBA 10 + CBD 3.8 + MLT 400 * | 0 | 0 |
| AKBA 10 + CBD 3.8 + MLT 800 * | 0 | 0 |
| AKBA 10 + CBD 7.6 + MLT 200 * | 0 | 0 |
| AKBA 10 + CBD 7.6 + MLT 400 * | 0 | 0 |
| AKBA 10 + CBD 7.6 + MLT 800 * | 0 | 0 |
| AKBA 10 + CBD 15.2 + MLT 200 * | 0 | 0 |
| AKBA 10 + CBD 15.2 + MLT 400 * | 0 | 0 |
| AKBA 10 + CBD 15.2 + MLT 800 * | 0 | 0 |
| AKBA 15 + CBD 3.8 + MLT 200 * | 0 | 0 |
| AKBA 15 + CBD 3.8 + MLT 400 * | 0 | 0 |
| AKBA 15 + CBD 3.8 + MLT 800 * | 0 | 0 |
| AKBA 15 + CBD 7.6 + MLT 200 * | 0 | 0 |
| AKBA 15 + CBD 7.6 + MLT 400 * | 0 | 0 |
| AKBA 15 + CBD 7.6 + MLT 800 * | 0 | 0 |
| AKBA 15 + CBD 15.2 + MLT 200 * | 0 | 0 |
| AKBA 15 + CBD 15.2 + MLT 400 * | 0 | 0 |
| AKBA 15 + CBD 15.2 + MLT 800 * | 0 | 0 |

The data are representative of three independent experiments.
The cell viability was represented as Average ± SD.
Table 2 includes data of cell viability (% vs vehicle) as a function of dose (μgrams) showing that CBD, MLT and AKBA induced cytotoxicity in PDAC cell lines PANC-1 and MIAPaCa-2 according to exemplary embodiments of the present disclosure.
Cell lines were treated with daily administration of different doses of the compounds as indicated on the Table 2.
Cell viability was evaluated at 72 hours post-treatment, by MTT assay.
Data shown are expressed as mean ± SE of three separate experiments and six wells for each dose.
Synergism was indicated with an*, such that a pharmacological level, the combinations are more effective than the single compounds, so maybe these effects can improve the importance to use the combination respect to single compounds.

CBD conversion: 6.25 μM=1.9 mg/ml; 25 μM=3.8 mg/ml; 50 μM=7.6 mg/ml; 100 μM=15.2 mg/ml;

So, considering the different doses used the effective application range is (AKBA:CBD:MLT; micrograms): from (all range of combination). CBD from 1 microgram; MLT from 10 micrograms; AKBA from 1 microgram

### Gene Expression Results

To elucidate the molecular events induced by CBD, MLT and AKBA alone and in combination, 92 genes involved in PDAC progression and aggressiveness were evaluated by Taqman Array. Both cell lines were treated with CBD, MLT and AKBA alone and in combination, and the molecular pathways of PDAC associated gene were evaluated. As shown (TABLE GENE EXPRESSION, Tables below are referred to gene expression analysis in PANC-1 and MIAPaCa-2 cell lines), different pathways involved in PDAC carcinogenesis were modulated by CBD, MLT, AKBA alone and in combination, suggesting that both treatments influence common but also specific pathways (Table 3, 4) shown in below and in Appendix 5 and 6.

In particular, the combination data provides synergistic results and the combinations amplified the effects on gene expression.

TABLE 3

| | | | | | | | | | CBD − |
|---|---|---|---|---|---|---|---|---|---|
| | | | | | CBD + + | | CBD − | MLT − | MLT − |
| | | VEI | CBD | MLT | MLT | AKBA | AKBA | AKBA | AKBA |
| PANC-1 | GAPDH | 1.00 | 1.00 | 1.00 | 1.00 | 1.00 | 1.00 | 1.00 | 1.00 |
| elative expression | | | | | | | | | |
| Akt-pathways | AKT1 | 0.11 | 0.09 | 0.10 | 0.08 | 0.20 | 0.12 | 0.12 | 0.15 |
| Akt-pathways | AKT2 | 0.50 | 0.45 | 0.46 | 0.40 | 0.41 | 0.44 | 0.44 | 0.66 |
| Akt-pathways | AKT3 | 0.00 | 0.00 | 0.00 | 0.00 | 0.00 | 0.00 | 0.00 | 0.01 |
| Ras pathways | ARHGEF7 | 0.00 | 0.00 | 0.017 | 0.02 | 0.00 | 0.00 | 0.00 | 0.02 |
| apoptosis | BCL2 | 0.00 | 0.00 | 0.00 | 0.00 | 0.00 | 0.00 | 0.00 | 0.00 |
| apoptosis | BCL2L1 | 0.35 | 0.21 | 0.21 | 0.26 | 0.23 | 0.18 | 0.14 | 0.41 |
| apoptosis | BIRC5 | 0.23 | 0.09 | 0.13 | 0.08 | 0.22 | 0.10 | 0.11 | 0.08 |
| Ras pathways | BRAF | 0.22 | 0.18 | 0.13 | 0.10 | 0.18 | 0.14 | 0.11 | 0.00 |
| DNA repair | BRCA2 | 0.03 | 0.13 | 0.34 | 3.72 | 0.10 | 0.23 | 0.40 | 0.05 |
| cell cycle | CCNA2 | 0.07 | 0.01 | 0.07 | 0.01 | 0.10 | 0.01 | 0.01 | 0.05 |
| cell cycle | CCNB1 | 0.27 | 0.31 | 0.33 | 0.30 | 0.20 | 0.30 | 0.30 | 0.17 |
| cell cycle | CCND1 | 0.06 | 0.09 | 0.10 | 0.06 | 0.10 | 0.08 | 0.08 | 0.10 |
| cell cycle | CCND2 | 0.00 | 0.00 | 0.00 | 0.04 | 0.00 | 0.00 | 0.00 | 0.00 |
| cell cycle | CCNE1 | 0.09 | 0.08 | 0.11 | 0.09 | 0.10 | 0.09 | 0.09 | 0.10 |
| cell cycle | CCNE2 | 0.07 | 0.10 | 0.07 | 0.06 | 0.06 | 0.08 | 0.08 | 0.06 |
| cell cycle | CDC42 | 0.45 | 0.04 | 0.00 | 0.00 | 0.02 | 0.02 | 0.02 | 0.00 |
| cell cycle | CDK2 | 0.09 | 0.13 | 0.07 | 0.06 | 0.10 | 0.09 | 0.09 | 0.08 |
| cell cycle | CDK4 | 0.24 | 0.21 | 0.17 | 0.18 | 0.25 | 0.19 | 0.19 | 0.20 |
| cell cycle | CDKN1A | 0.49 | 0.03 | 0.07 | 0.10 | 0.28 | 0.01 | 0.01 | 0.14 |
| cell cycle | CDKN1B | 0.10 | 0.09 | 0.09 | 0.11 | 0.10 | 0.08 | 0.08 | 0.15 |
| cell cycle | CDKN2A | 0.10 | 0.22 | 0.16 | 0.23 | 0.12 | 0.34 | 0.32 | 0.00 |
| cell cycle | CDKN2B | 0.00 | 0.35 | 1.65 | 1.71 | 0.20 | 0.65 | 0.88 | 0.00 |
| cell cycle | CDKN2C | 0.04 | 0.03 | 0.14 | 0.45 | 0.12 | 0.17 | 0.72 | 0.02 |
| cell cycle | CDKN2D | 0.03 | 0.03 | 0.04 | 0.14 | 0.05 | 0.14 | 0.16 | 0.05 |
| cytochrome p450 | CYP2E1 | 0.00 | 0.00 | 0.00 | 0.01 | 0.00 | 0.00 | 0.00 | 0.00 |
| Ras pathways | EZF1 | 0.17 | 0.13 | 0.14 | 0.13 | 0.14 | 0.11 | 0.11 | 0.13 |
| Ras pathways | EZF3 | 0.07 | 0.07 | 0.08 | 0.08 | 0.07 | 0.07 | 0.07 | 0.09 |
| Ras pathways | EZF4 | 0.16 | 0.16 | 0.16 | 0.20 | 0.18 | 0.18 | 0.14 | 0.19 |
| Ras pathways | EGF | 0.00 | 0.00 | 0.00 | 0.00 | 0.00 | 0.00 | 0.00 | 0.08 |
| Ras pathways | EGFR | 0.24 | 0.17 | 0.16 | 0.14 | 0.18 | 0.11 | 0.13 | 0.19 |
| Ras pathways | ELK1 | 0.41 | 0.07 | 0.09 | 0.09 | 0.11 | 0.08 | 0.06 | 0.10 |
| Ras pathways | ERBB2 | 0.27 | 0.01 | 0.04 | 0.03 | 0.07 | 0.02 | 0.02 | 0.02 |
| angiogenesis | FIGF | 0.14 | 0.09 | 0.11 | 0.07 | 0.00 | 0.08 | 0.07 | 0.00 |
| Ras pathways | GRB2 | 0.31 | 0.11 | 0.23 | 0.13 | 0.38 | 0.12 | 0.22 | 0.19 |
| Ras pathways | HBEGF | 0.53 | 0.11 | 0.39 | 0.45 | 0.44 | 0.11 | 0.27 | 0.39 |
| Heat sock protein | HSP90AA1 | 0.35 | 0.35 | 0.00 | 0.00 | 0.22 | 0.28 | 0.00 | 0.39 |
| Akt-pathways | IGF1 | 0.00 | 0.00 | 0.00 | 0.00 | 0.00 | 0.00 | 0.00 | 0.00 |
| inflammation | IL6 | 0.00 | 0.00 | 0.00 | 0.00 | 0.00 | 0.00 | 0.00 | 0.00 |
| inflammation | JAK1 | 0.23 | 0.22 | 0.02 | 0.02 | 1.10 | 0.78 | 0.13 | 0.34 |
| inflammation | JAK2 | 0.01 | 0.01 | 0.01 | 0.01 | 0.04 | 0.01 | 0.01 | 0.03 |
| inflammation | JAK3 | 0.01 | 0.01 | 0.00 | 0.00 | 0.03 | 0.00 | 0.00 | 0.01 |
| angiogenesis | KDR | 0.00 | 0.00 | 0.00 | 0.00 | 0.00 | 0.00 | 0.00 | 0.00 |
| proliferation | KIT | 0.00 | 0.00 | 0.01 | 0.01 | 0.00 | 0.00 | 0.00 | 0.00 |
| Ras pathways | KRAS | 0.09 | 0.07 | 0.01 | 0.00 | 0.09 | 0.04 | 0.04 | 0.06 |
| Ras pathways | MAP2K1 | 0.08 | 0.00 | 0.03 | 0.02 | 0.10 | 0.01 | 0.01 | 0.17 |
| Ras pathways | MAP2K2 | 0.06 | 0.07 | 0.08 | 0.05 | 0.10 | 0.06 | 0.05 | 0.10 |
| Ras pathways | MAPK1 | 0.05 | 0.06 | 0.02 | 0.01 | 0.60 | 0.22 | 0.31 | 0.12 |
| Ras pathways | MAPK3 | 0.00 | 0.01 | 0.00 | 0.01 | 0.00 | 0.00 | 0.00 | 0.03 |
| DNA repair | MDM2 | 0.03 | 0.04 | 0.00 | 0.00 | 0.04 | 0.02 | 0.02 | 0.08 |
| invasion | MMP1 | 0.00 | 0.00 | 0.07 | 0.05 | 0.02 | 0.02 | 0.02 | 0.02 |
| invasion | MMP2 | 0.08 | 0.06 | 0.00 | 0.00 | 0.20 | 0.14 | 0.07 | 0.06 |
| invasion | MMP3 | 0.00 | 0.00 | 0.00 | 0.00 | 0.00 | 0.00 | 0.00 | 0.00 |
| invasion | MMP7 | 0.01 | 0.01 | 0.01 | 0.01 | 0.03 | 0.00 | 0.00 | 0.00 |
| invasion | MMP9 | 0.01 | 0.01 | 0.04 | 0.04 | 0.03 | 0.00 | 0.00 | 0.00 |
| NfkB pathways | NFKB1 | 0.35 | 0.04 | 0.09 | 0.03 | 0.10 | 0.08 | 0.08 | 0.06 |
| NfkB pathways | NFKB2 | 0.16 | 0.12 | 0.02 | 0.00 | 0.10 | 0.05 | 0.00 | 0.15 |
| proliferation | NOTCH1 | 1.03 | 0.02 | 0.32 | 0.03 | 0.05 | 0.12 | 0.00 | 0.04 |
| Akt-pathways | PK3CA | 0.01 | 0.02 | 0.00 | 0.01 | 0.02 | 0.00 | 0.00 | 0.03 |
| Akt-pathways | PK3CB | 0.11 | 0.08 | 0.13 | 0.08 | 0.16 | 0.08 | 0.08 | 0.13 |
| Akt-pathways | PK3CD | 0.06 | 0.07 | 0.06 | 0.05 | 0.05 | 0.06 | 0.06 | 0.13 |
| Akt-pathways | PK3R1 | 0.04 | 0.03 | 0.02 | 0.03 | 0.03 | 0.03 | 0.03 | 0.07 |
| Akt-pathways | PK3R2 | 0.11 | 0.10 | 0.00 | 0.00 | 0.13 | 0.07 | 0.08 | 0.17 |
| invasion | PTGS2 | 0.04 | 0.00 | 0.02 | 0.01 | 0.08 | 0.04 | 0.04 | 0.01 |
| invasion | RAC1 | 0.09 | 0.09 | 0.00 | 0.01 | 0.10 | 0.05 | 0.05 | 0.11 |
| invasion | RAC2 | 0.01 | 0.01 | 0.01 | 0.01 | 0.01 | 0.00 | 0.00 | 0.00 |
| Ras pathways | RAF1 | 0.25 | 0.01 | 0.06 | 0.09 | 0.08 | 0.00 | 0.00 | 0.03 |
| Ras pathways | RB1 | 0.01 | 0.05 | 0.03 | 0.03 | 0.15 | 0.22 | 0.23 | 0.08 |
| NfkB pathways | REL | 0.09 | 0.10 | 0.08 | 0.00 | 0.08 | 0.07 | 0.07 | 0.03 |
| NfkB pathways | RELA | 0.10 | 0.06 | 0.00 | 0.01 | 0.10 | 0.06 | 0.06 | 0.09 |
| NfkB pathways | RELB | 0.42 | 0.23 | 0.06 | 0.05 | 0.12 | 0.08 | 0.08 | 0.05 |
| NfkB pathways | RHOA | 0.37 | 0.30 | 0.07 | 0.07 | 0.30 | 0.28 | 0.28 | 0.39 |
| NfkB pathways | RHOB | 0.03 | 0.03 | 0.03 | 0.02 | 0.05 | 0.03 | 0.03 | 0.05 |
| TGF-patways | SMAD2 | 0.05 | 0.06 | 0.19 | 0.13 | 0.04 | 0.09 | 0.08 | 0.09 |
| TGF-patways | SMAD3 | 0.17 | 0.16 | 0.22 | 0.88 | 0.20 | 0.18 | 0.28 | 0.11 |
| TGF-patways | SMAD4 | 0.07 | 0.07 | 0.08 | 0.09 | 0.09 | 0.12 | 0.12 | 0.10 |
| Ras pathways | SOS1 | 0.07 | 0.06 | 0.06 | 0.04 | 0.10 | 0.05 | 0.05 | 0.15 |
| proliferation | SRC | 0.27 | 0.03 | 0.03 | 0.04 | 0.14 | 0.03 | 0.03 | 0.05 |
| inflammation | STAT1 | 0.18 | 0.10 | 0.12 | 0.06 | 0.31 | 0.22 | 0.24 | 0.12 |
| inflammation | STAT2 | 0.09 | 0.03 | 0.00 | 0.00 | 0.11 | 0.09 | 0.09 | 0.05 |
| inflammation | STAT3 | 4.45 | 0.05 | 0.07 | 0.05 | 0.18 | 0.04 | 0.04 | 0.07 |
| inflammation | STATSB | 0.04 | 0.03 | 0.45 | 0.00 | 0.20 | 0.03 | 0.03 | 0.10 |
| inflammation | STAT6 | 1.38 | 0.87 | 0.01 | 0.00 | 0.13 | 0.06 | 0.06 | 0.05 |
| TGF-patways | TGFA | 0.00 | 0.71 | 0.22 | 0.18 | 0.02 | 0.68 | 0.28 | 0.00 |
| TGF-patways | TGFB1 | 0.07 | 0.07 | 0.05 | 0.02 | 0.15 | 0.05 | 0.05 | 0.11 |
| TGF-patways | TGFB2 | 0.00 | 0.08 | 0.00 | 0.00 | 0.12 | 0.12 | 0.02 | 0.03 |
| TGF-patways | TGFB3 | 0.01 | 0.00 | 0.00 | 0.00 | 0.05 | 0.00 | 0.00 | 0.01 |
| TGF-patways | TGFBR1 | 0.02 | 0.03 | 0.00 | 0.01 | 0.07 | 0.08 | 0.08 | 0.06 |
| TGF-patways | TGFBR2 | 0.06 | 0.01 | 0.00 | 0.29 | 0.10 | 0.11 | 0.10 | 0.06 |
| DNA repair | TP53 | 0.14 | 0.24 | 0.29 | 0.27 | 0.38 | 0.44 | 0.55 | 0.34 |
| angiogenesis | VEGFA | 0.06 | 0.03 | 0.03 | 0.00 | 0.45 | 0.44 | 0.45 | 0.19 |
| angiogenesis | VEGFB | 0.06 | 0.07 | 0.05 | 0.00 | 0.18 | 0.22 | 0.27 | 0.08 |
| angiogenesis | VEGFC | 0.08 | 0.03 | 0.00 | 0.00 | 0.12 | 0.13 | 0.13 | 0.04 |

TABLE 4

| | | | | | | | | | CBD − |
|---|---|---|---|---|---|---|---|---|---|
| | | | | | CBD + + | | CBD − | MLT − | MLT − |
| | | VEI | CBD | MLT | MLT | AKBA | AKBA | AKBA | AKBA |
| Mia-PACA-2 | GAPDH | 1.00 | 1.00 | 1.00 | 1.00 | 1.00 | 1.00 | 1.00 | 1.00 |
| relative expression | | | | | | | | | |
| Akt-pathways | AKT1 | 0.13 | 0.10 | 0.09 | 0.07 | 0.18 | 0.11 | 0.11 | 0.11 |
| Akt-pathways | AKT2 | 0.60 | 0.54 | 0.55 | 0.49 | 0.49 | 0.53 | 0.53 | 0.50 |
| Akt-pathways | AKT3 | 0.00 | 0.00 | 0.00 | 0.00 | 0.00 | 0.00 | 0.00 | 0.00 |
| Ras pathways | ARHGEF7 | | | | | | | | |
| apoptosis | BCL2 | 0.00 | 0.00 | 0.00 | 0.00 | 0.00 | 0.00 | 0.00 | 0.00 |
| apoptosis | BCL2L1 | 0.42 | 0.26 | 0.26 | 0.31 | 0.28 | 0.22 | 0.17 | 0.14 |
| apoptosis | BIRC5 | 0.27 | 0.11 | 0.16 | 0.10 | 0.26 | 0.12 | 0.13 | 0.10 |
| Ras pathways | BRAF | 0.26 | 0.22 | 0.16 | 0.12 | 0.22 | 0.17 | 0.13 | 0.12 |
| DNA repair | BRCA2 | 0.04 | 0.16 | 0.40 | 4.46 | 0.12 | 0.28 | 0.48 | 0.38 |
| cell cycle | CCNA2 | 0.09 | 0.01 | 0.09 | 0.01 | 0.12 | 0.01 | 0.01 | 0.01 |
| cell cycle | CCNB1 | 0.33 | 0.37 | 0.40 | 0.36 | 0.24 | 0.36 | 0.36 | 0.29 |
| cell cycle | CCND1 | 0.10 | 0.11 | 0.12 | 0.09 | 0.12 | 0.10 | 0.10 | 0.10 |
| cell cycle | CCND2 | 0.00 | 0.00 | 0.00 | 0.04 | 0.00 | 0.00 | 0.00 | 0.00 |
| cell cycle | CCNE1 | 0.11 | 0.10 | 0.13 | 0.11 | 0.12 | 0.11 | 0.11 | 0.07 |
| cell cycle | CCNE2 | 0.08 | 0.12 | 0.09 | 0.07 | 0.10 | 0.10 | 0.10 | 0.10 |
| cell cycle | CDC42 | 0.54 | 0.05 | 0.00 | 0.00 | 0.02 | 0.02 | 0.02 | 0.02 |
| cell cycle | CDK2 | 0.11 | 0.15 | 0.09 | 0.08 | 0.12 | 0.11 | 0.11 | 0.10 |
| cell cycle | CDK4 | 0.29 | 0.25 | 0.20 | 0.21 | 0.30 | 0.23 | 0.23 | 0.14 |
| cell cycle | CDKN1A | 0.59 | 0.04 | 0.08 | 0.12 | 0.34 | 0.01 | 0.01 | 0.01 |
| cell cycle | CDKN1B | 0.12 | 0.11 | 0.11 | 0.13 | 0.12 | 0.10 | 0.10 | 0.17 |
| cell cycle | CDKN2A | 0.12 | 0.26 | 0.20 | 0.27 | 0.14 | 0.41 | 0.38 | 0.36 |
| cell cycle | CDKN2B | 0.00 | 0.42 | 1.98 | 2.05 | 0.24 | 0.78 | 0.88 | 2.32 |
| cell cycle | CDKN2C | 0.04 | 0.03 | 0.17 | 0.54 | 0.14 | 0.20 | 0.86 | 0.94 |
| cell cycle | CDKN2D | 0.04 | 0.04 | 0.05 | 0.17 | 0.06 | 0.17 | 0.19 | 0.14 |
| cytochrome p450 | CYP2E1 | | | | | | | | |
| Ras pathways | E2F1 | 0.20 | 0.15 | 0.16 | 0.15 | 0.17 | 0.13 | 0.13 | 0.12 |
| Ras pathways | E2F3 | 0.08 | 0.08 | 0.09 | 0.10 | 0.08 | 0.08 | 0.08 | 0.08 |
| Ras pathways | E2F4 | 0.19 | 0.19 | 0.19 | 0.24 | 0.22 | 0.22 | 0.17 | 0.16 |
| Ras pathways | EGF | 0.00 | 0.00 | 0.00 | 0.00 | 0.00 | 0.00 | 0.00 | 0.00 |
| Ras pathways | EGFR | 0.28 | 0.21 | 0.19 | 0.17 | 0.22 | 0.13 | 0.16 | 0.13 |
| Ras pathways | ELK1 | 0.49 | 0.09 | 0.11 | 0.11 | 0.13 | 0.10 | 0.07 | 0.07 |
| Ras pathways | ERBB2 | 0.33 | 0.02 | 0.05 | 0.03 | 0.08 | 0.02 | 0.02 | 0.02 |
| angiogenesis | FIGF | 0.17 | 0.11 | 0.14 | 0.09 | 0.00 | 0.10 | 0.08 | 0.01 |
| Ras pathways | GRB2 | 0.37 | 0.13 | 0.28 | 0.15 | 0.46 | 0.14 | 0.26 | 0.19 |
| Ras pathways | HBEGF | 0.64 | 0.13 | 0.46 | 0.54 | 0.53 | 0.13 | 0.32 | 0.25 |
| Heat sock protein | HSP90AA1 | 0.42 | 0.42 | 0.00 | 0.00 | 0.26 | 0.34 | 0.00 | 0.00 |
| Akt-pathways | IGF1 | 0.00 | 0.00 | 0.00 | 0.00 | 0.00 | 0.00 | 0.00 | 0.00 |
| inflammation | IL6 | 0.00 | 0.00 | 0.00 | 0.00 | 0.00 | 0.00 | 0.00 | 0.00 |
| inflammation | JAK1 | 0.28 | 0.27 | 0.02 | 0.02 | 1.32 | 0.94 | 0.16 | 0.52 |
| inflammation | JAK2 | 0.01 | 0.01 | 0.02 | 0.01 | 0.05 | 0.01 | 0.01 | 0.01 |
| inflammation | JAK3 | 0.01 | 0.01 | 0.00 | 0.00 | 0.04 | 0.00 | 0.00 | 0.00 |
| angiogenesis | KDR | | | | | | | | |
| proliferation | KIT | 0.01 | 0.00 | 0.01 | 0.01 | 0.00 | 0.00 | 0.00 | 0.00 |
| Ras pathways | KRAS | 0.11 | 0.09 | 0.01 | 0.00 | 0.11 | 0.05 | 0.05 | 0.05 |
| Ras pathways | MAP2K1 | 0.10 | 0.00 | 0.04 | 0.03 | 0.12 | 0.01 | 0.01 | 0.01 |
| Ras pathways | MAP2K2 | 0.07 | 0.08 | 0.09 | 0.06 | 0.12 | 0.07 | 0.07 | 0.07 |
| Ras pathways | MAPK1 | 0.06 | 0.07 | 0.03 | 0.01 | 0.72 | 0.26 | 0.37 | 0.32 |
| Ras pathways | MAPK3 | 0.01 | 0.01 | 0.01 | 0.01 | 0.00 | 0.00 | 0.00 | 0.00 |
| DNA repair | MDM2 | 0.04 | 0.04 | 0.00 | 0.00 | 0.05 | 0.02 | 0.02 | 0.02 |
| invasion | MMP1 | 0.00 | 0.00 | 0.08 | 0.06 | 0.02 | 0.02 | 0.02 | 0.02 |
| invasion | MMP2 | 0.09 | 0.07 | 0.00 | 0.00 | 0.24 | 0.17 | 0.08 | 0.10 |
| invasion | MMP3 | 0.00 | 0.00 | 0.01 | 0.01 | 0.00 | 0.00 | 0.00 | 0.00 |
| invasion | MMP7 | 0.01 | 0.01 | 0.01 | 0.01 | 0.04 | 0.00 | 0.00 | 0.00 |
| invasion | MMP9 | 0.01 | 0.01 | 0.04 | 0.05 | 0.04 | 0.00 | 0.00 | 0.00 |
| NfkB pathways | NFKB1 | 0.41 | 0.04 | 0.11 | 0.04 | 0.12 | 0.10 | 0.10 | 0.10 |
| NfkB pathways | NFKB2 | 0.20 | 0.15 | 0.02 | 0.00 | 0.12 | 0.06 | 0.00 | 0.00 |
| proliferation | NOTCH1 | 1.23 | 0.02 | 0.38 | 0.04 | 0.06 | 0.14 | 0.00 | 0.00 |
| Akt-pathways | PK3CA | 0.01 | 0.03 | 0.00 | 0.01 | 0.02 | 0.00 | 0.00 | 0.00 |
| Akt pathways | PK3CB | 0.13 | 0.10 | 0.15 | 0.10 | 0.19 | 0.10 | 0.10 | 0.08 |
| Akt pathways | PK3CD | 0.07 | 0.08 | 0.07 | 0.06 | 0.06 | 0.07 | 0.07 | 0.07 |
| Akt-pathways | PK3R1 | 0.05 | 0.04 | 0.03 | 0.04 | 0.04 | 0.04 | 0.04 | 0.04 |
| Akt-pathways | PK3R2 | 0.14 | 0.12 | 0.00 | 0.01 | 0.16 | 0.08 | 0.10 | 0.04 |
| invasion | PTGS2 | 0.05 | 0.00 | 0.03 | 0.01 | 0.10 | 0.05 | 0.05 | 0.05 |
| invasion | RAC1 | 0.11 | 0.11 | 0.00 | 0.01 | 0.12 | 0.06 | 0.06 | 0.06 |
| invasion | RAC2 | 0.01 | 0.01 | 0.01 | 0.01 | 0.01 | 0.00 | 0.00 | 0.00 |
| Ras pathways | RAF1 | 0.30 | 0.01 | 0.07 | 0.11 | 0.10 | 0.00 | 0.00 | 0.00 |
| Ras pathways | RB1 | 0.01 | 0.06 | 0.03 | 0.03 | 0.17 | 0.24 | 0.25 | 0.23 |
| NfkB pathways | REL | 0.10 | 0.11 | 0.09 | 0.00 | 0.09 | 0.08 | 0.08 | 0.08 |
| NfkB pathways | RELA | 0.11 | 0.06 | 0.00 | 0.02 | 0.11 | 0.07 | 0.07 | 0.07 |
| NfkB pathways | RELB | 0.47 | 0.26 | 0.06 | 0.05 | 0.13 | 0.09 | 0.09 | 0.06 |
| NfkB pathways | RHOA | 0.41 | 0.34 | 0.07 | 0.08 | 0.33 | 0.31 | 0.31 | 0.23 |
| NfkB pathways | RHOB | 0.04 | 0.04 | 0.03 | 0.02 | 0.06 | 0.03 | 0.03 | 0.03 |
| TGF-patways | SMAD2 | 0.06 | 0.06 | 0.21 | 0.14 | 0.04 | 0.10 | 0.09 | 0.09 |
| TGF-patways | SMAD3 | 0.19 | 0.18 | 0.24 | 0.97 | 0.22 | 0.20 | 0.31 | 0.36 |
| TGF-patways | SMAD4 | 0.08 | 0.07 | 0.09 | 0.10 | 0.10 | 0.13 | 0.13 | 0.15 |
| Ras pathways | SOS1 | 0.07 | 0.06 | 0.07 | 0.05 | 0.11 | 0.06 | 0.06 | 0.06 |
| proliferation | SRC | 0.29 | 0.03 | 0.07 | 0.05 | 0.15 | 0.03 | 0.03 | 0.03 |
| inflammation | STAT1 | 0.20 | 0.11 | 0.13 | 0.06 | 0.34 | 0.24 | 0.26 | 0.23 |
| inflammation | STAT2 | 0.10 | 0.04 | 0.00 | 0.00 | 0.12 | 0.10 | 0.10 | 0.10 |
| inflammation | STAT3 | 4.89 | 0.06 | 0.08 | 0.05 | 0.20 | 0.04 | 0.04 | 0.04 |
| inflammation | STATSB | 0.05 | 0.03 | 0.49 | 0.00 | 0.22 | 0.03 | 0.03 | 0.03 |
| inflammation | STAT6 | 1.24 | 0.79 | 0.01 | 0.00 | 0.12 | 0.05 | 0.05 | 0.05 |
| TGF-patways | TGFA | 0.00 | 0.64 | 0.20 | 0.16 | 0.02 | 0.61 | 0.25 | 0.30 |
| TGF-patways | TGFB1 | 0.06 | 0.06 | 0.04 | 0.02 | 0.13 | 0.05 | 0.05 | 0.05 |
| TGF patways | TGFB2 | 0.00 | 0.08 | 0.00 | 0.00 | 0.10 | 0.11 | 0.02 | 0.02 |
| TGF patways | TGFB3 | 0.01 | 0.00 | 0.00 | 0.00 | 0.05 | 0.00 | 0.00 | 0.00 |
| TGF patways | TGFBR1 | 0.02 | 0.02 | 0.00 | 0.01 | 0.06 | 0.07 | 0.07 | 0.07 |
| TGF-patways | TGFBR2 | 0.06 | 0.00 | 0.00 | 0.26 | 0.09 | 0.10 | 0.09 | 0.12 |
| DNA repair | TP53 | 0.13 | 0.22 | 0.26 | 0.24 | 0.35 | 0.40 | 0.50 | 0.56 |
| angiogenesis | VEGFA | 0.05 | 0.02 | 0.02 | 0.00 | 0.41 | 0.40 | 0.41 | 0.59 |
| angiogenesis | VEGFB | 0.06 | 0.06 | 0.04 | 0.00 | 0.16 | 0.20 | 0.24 | 0.22 |
| angiogenesis | VEGFC | 0.07 | 0.03 | 0.00 | 0.00 | 0.11 | 0.12 | 0.12 | 0.14 |

TABLE 3 and 4: Modulation of PDAC pathways in PANC-1 and MIAPaCa-2 cell lines. mRNA expression was evaluated by TaqMan array in the cell lines, treated for 24 hs with CBD (3.8 micrograms/ml), MLT (200 micrograms/ml), AKBA (7.5 micrograms/ml) alone and in combination. Target mRNA levels were normalized for GAPDH expression. Table 3 and 4 includes data from TaqMan® Array Human Pancreatic Adenocarcinoma 96-well Plate, containing 92 assays to pancreatic adenocarcinoma associated genes.

Regarding gene expression analysis in PANC-1 and MIAPaCa-2 cell line, the effect of CBD+MLT+AKBA compared to vehicle-treated cells was:

Regarding the effect in regulating genes involved in Ras pathway, CBD, MLT and AKBA in combination, were able in down-regulating EGFR, ELK1, ERBB2, FIGF, GRB2, HBEGF, E2F1, RAF-1.

Regarding mitogenic pathways, was evidenced that CBD, MLT and AKBA in combination significantly down-regulate CDC42, CDK4, SRC, while RB1, CDKN2A, CDKN2B, CDKN2C were up-regulated.

Regarding DNA repair pathways, was also detected that CBD, MLT and AKBA in combination significantly increases the TP53, BRCA2 gene, which are associated with DNA repair.

Regarding the Nf-kB pathway, CBD, MLT and AKBA in combination, reduced NFKB1 NFKB2, RELB, RHOA gene expression.

Regarding Phosphatidylinositol 3-kinase/Protein kinase B (PI3K/AKT) pathway, CBD, MLT and AKBA in combination, reduced PK3R2 gene expression.

Regarding VEGF pathways, CBD, MLT and AKBA in combination, increased VEGFA and VEGFB.

Regarding TGF-beta pathways, CBD, MLT and AKBA in combination, increased the gene expression of TGFA, TGFBR1, TGFBR2, SMAD3, SMAD4.

While the disclosure has been described with reference to what are presently considered to be the preferred examples, it is to be understood that the disclosure is not limited to the disclosed examples. To the contrary, the present disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### Route of Administration

CBD can be used as crystal, in oil, ethanol, soluble form (in the marked there are soluble CBDs), capsules, vaginal eggs, suppositories.

MLT is soluble in: water, ethanol, benzene, chloroform, methanol, DMSO, toluene, and dilute aqueous acid, and very slightly soluble in petroleum ether, and used as for CBD. Also for melatonin in the marked there are soluble forms and conjugated-forms (Melatonin with adenosine solubilized in water and stabilized with glycine for oncological treatment). AKBA is sparingly soluble in aqueous buffers. For maximum solubility in aqueous buffers, AKBA should first be dissolved in DMSO and then diluted with the aqueous buffer of choice. AKBA has a solubility of approximately 0.3 mg/ml in a 1:2 solution of DMSO:PBS (pH 7.2). Moreover, AKBA can be obtained from the stem of the tree B. serrata (frankincense) and probably from others vegetal sources.

The combinations were effective in other human tumor cell lines and we tested in glioblastoma cell line and endometrial cancer cell Additional tests include data obtained from two independent experiments, six wells for dose.

TABLE 5
Cytotoxic effects of CBD, MLT and AKBA alone and in combination in U251
Glioblastoma cell line and PECM004a endometrial cancer cell line.

| | U251 | PCEM004a |
|---|---|---|
| | CELL LINE | CELL LINE |
| | (% VIABILITY ± | (% VIABILITY ± |
| COMPOUNDS (micrograms/ml) | S.D.) | S.D.) |
| AKBA 5 | 100 ± 5.3 | 100 ± 7.5 |
| AKBA 7.5 | 100 ± 6.6 | 98.4 ± 6.3 |
| AKBA 10 | 100 ± 2.2 | 87.4 ± 5.8 |
| AKBA 15 | 76.4 ± 4.2 | 60.2 ± 5.4 |
| CBD 1.9 | 100 ± 7.1 | 74.8 ± 7.2 |
| CBD 3.8 | 100 ± 5.5 | 93.2 ± 4.2 |
| CBD 7.6 | 97 ± 10.1 | 93.4 ± 5.4 |
| CBD 15.2 | 65.2 ± 4.3 | 50.4 ± 3.4 |
| MLT 100 | 100 ± 2.3 | 96.5 ± 8.2 |
| MLT 200 | 65.4 ± 4.4 | 90.4 ± 6.2 |
| MLT 400 | 58.2 ± 4.1 | 80.1 ± 5.3 |
| MLT 800 | 17.0 ± 1.1 | 31.3 ± 2.8 |
| AKBA 5 + CBD 1.9 | 92.3 ± 6.5 | 75.8 ± 6.3 |
| AKBA 7.5 + CBD 1.9 | 87.1 ± 5.9 | 66.4 ± 4.1 |
| AKBA 10 + CBD 1.9 | 92.2 ± 7.2 | 74.4 ± 5.9 |
| AKBA 15 + CBD 1.9 | 61.4 ± 5.7 | 44.4 ± 3.1 |
| AKBA 5 + CBD 3.8 | 80.2 ± 6.6 | 65.3 ± 5.8 |
| AKBA 7.5 + CBD 3.8 | 70.7 ± 6.1 | 52.4 ± 4.1 |
| AKBA 10 + CBD 3.8 | 75.5 ± 5.4 | 46.6 ± 3.6 |
| AKBA 15 + CBD 3.8 | 63.3 ± 4.2 | 41.4 ± 3.1 |
| AKBA 5 + CBD 7.6 * | 33.4 ± 2.4 | 16.2 ± 1.4 |
| AKBA 7.5 + CBD 7.6 * | 100 ± 0.5 | 93.4 ± 7.2 |
| AKBA 10 + CBD 7.6 * | 44.2 ± 2.2 | 36.6 ± 3.2 |
| AKBA 15 + CBD 7.6 * | 27.9 ± 2.3 | 33.2 ± 2.1 |
| AKBA 5 + CBD 15.2 * | 25.9 ± 1.1 | 22.4 ± 1.1 |
| AKBA 7.5 + CBD 15.2 * | 16.5 ± 0.9 | 12.9 ± 0.7 |
| AKBA 10 + CBD 15.2 * | 12.3 ± 1.2 | 10.6 ± 1.9 |
| AKBA 15 + CBD 15.2 * | 5.6 ± 0.1 | 0 |
| AKBA 5 + MLT 100 | 85.2 ± 3.4 | 70.2 ± 5.9 |
| AKBA 7.5 + MLT 100 | 89.3 ± 6.5 | 68.4 ± 5.5 |
| AKBA 10 + MLT 100 | 82.5 ± 4.2 | 65.4 ± 5.7 |
| AKBA 15 + MLT 100 * | 56.7 ± 4.1 | 39.5 ± 2.7 |
| AKBA 5 + MLT 200 | 82.2 ± 6.3 | 65.5 ± 6.1 |
| AKBA 7.5 + MLT 200 | 77.6 ± 4.7 | 53.2 ± 4.6 |
| AKBA 10 + MLT 200 | 84.2 ± 5.1 | 62.6 ± 5.2 |
| AKBA 15 + MLT 200 * | 56.2 ± 4.1 | 38.6 ± 3.6 |
| AKBA 5 + MLT 400 | 76.9 ± 6.1 | 61.2 ± 5.4 |
| AKBA 7.5 + MLT 400 | 88.2 ± 7.2 | 73.1 ± 5.6 |
| AKBA 10 + MLT 400 * | 75.5 ± 5.2 | 59.3 ± 3.5 |
| AKBA 15 + MLT 400 * | 60.4 ± 5.3 | 39.7 ± 2.1 |
| AKBA 5 + MLT 800 * | 30.2 ± 1.5 | 17.2 ± 1.3 |
| AKBA 7.5 + MLT 800 * | 10.2 ± 0.5 | 11.2 ± 1.1 |
| AKBA 10 + MLT 800 * | 8.2 ± 1.7 | 2.2 ± 0.1 |
| AKBA 15 + MLT 800 * | 3.7 ± 0.5 | 0 |
| CBD 1.9 + MLT 100 | 97.0 ± 6.2 | 73.4 ± 5.4 |
| CBD 1.9 + MLT 200 | 82.0 ± 4.9 | 64.2 ± 5.4 |
| CBD 1.9 + MLT 400 | 81.1 ± 5.9 | 65.3 ± 5.2 |
| CBD 1.9 + MLT 800 * | 12.23 ± 1.1 | 4.2 ± 1.5 |
| CBD 3.8 + MLT 100 | 65.4 ± 5.1 | 26.4 ± 2.9 |
| CBD 3.8 + MLT 200 * | 60.4 ± 5.1 | 35 . . . 5 ± 2.7 |
| CBD 3.8 + MLT 400 * | 49.2 ± 4.6 | 39.45 ± 2.9 |
| CBD 3.8 + MLT 800 * | 9.2 ± 0.5 | 3.5 ± 0.1 |
| CBD 7.6 + MLT 100 * | 3.2 ± 0.2 | 1.4 ± 0.1 |
| CBD 7.6 + MLT 200 * | 20.1 ± 1.5 | 0 |
| CBD 7.6 + MLT 400 * | 77.2 ± 5.3 | 56.9 ± 4.4 |
| CBD 7.6 + MLT 800 * | 6.2 ± 0.5 | 0 |
| CBD 15.2 + MLT 100 * | 30.4 ± 1.2 | 0 |
| CBD 15.2 + MLT 200 * | 22.24 ± 1.1 | 0 |
| CBD 15.2 + MLT 400 * | 10.6 ± 0.5 | 0 |
| CBD 15.2 + MLT 800 * | 0 | 0 |
| FOR TRIPLICATE COMPOSITIONS WE | | |
| USED ONLY COMBINATION OF MOST | | |
| EFFECTIVE DOSES | | |
| AKBA 7.5 + CBD 3.8 + MLT 200 * | 79.5 ± 6.6 | 34.2 ± 2.2 |
| AKBA 7.5 + CBD 3.8 + MLT 400 * | 69.4 ± 5.5 | 28.7 ± 1.2 |
| AKBA 7.5 + CBD 3.8 + MLT 800 * | 7.5 ± 3.6 | 4.5 ± 0.2 |
| AKBA 7.5 + CBD 7.6 + MLT 200 * | 78.3 ± 6.1 | 0 |
| AKBA 7.5 + CBD 7.6 + MLT 400 * | 65.9 ± 5.5 | 46.5 ± 3.8 |
| AKBA 7.5 + CBD 7.6 + MLT 800 * | 39.2 ± 2.6 | 24.1 ± 2.2 |
| AKBA 7.5 + CBD 15.2 + MLT 200 * | 24.5 ± 1.8 | 15.1 ± 0.2 |
| AKBA 7.5 + CBD 15.2 + MLT 400 * | 19.1 ± 0.6 | 8.5 ± 0.3 |
| AKBA 7.5 + CBD 15.2 + MLT 800 * | 0 | 0 |
| AKBA 10 + CBD 3.8 + MLT 200 * | 52.2 ± 4.6 | 31.7 ± 2.8 |
| AKBA 10 + CBD 3.8 + MLT 400 * | 31.6 ± 2.6 | 14.4 ± 0.2 |
| AKBA 10 + CBD 3.8 + MLT 800 * | 11.2 ± 1.6 | 4.1 ± 0.2 |
| AKBA 10 + CBD 7.6 + MLT 200 * | 51.4 ± 3.6 | 33.6 ± 2.9 |
| AKBA 10 + CBD 7.6 + MLT 400 * | 33.9 ± 3.2 | 16.2 ± 1.2 |
| AKBA 10 + CBD 7.6 + MLT 800 * | 11.6 ± 0.6 | 0 |
| AKBA 10 + CBD 15.2 + MLT 200 * | 13.4 ± 1.1 | 0 |
| AKBA 10 + CBD 15.2 + MLT 400 * | 6.9 ± 0.1 | 0 |
| AKBA 10 + CBD 15.2 + MLT 800 * | 0 | 0 |
| AKBA 15 + CBD 3.8 + MLT 200 * | 16.9 ± 1.3 | 3.1 ± 0.2 |
| AKBA 15 + CBD 38 + MLT 400 * | 11.2 ± 1.0 | 0 |
| AKBA 15 + CBD 3.8 + MLT 800 * | 2.2 ± 0.3 | 0 |
| AKBA 15 + CBD 7.6 + MLT 200 * | 13.4 ± 1.6 | 0 |
| AKBA 15 + CBD 7.6 + MLT 400 * | 9.1 ± 0.6 | 0 |
| AKBA 15 + CBD 7.6 + MLT 800 * | 0 | 0 |
| AKBA 15 + CBD 15.2 + MLT 200 * | 3.7 ± 0.4 | 0 |
| AKBA 15 + CBD 15.2 + MLT 400 * | 0 | 0 |
| AKBA 15 + CBD 15.2 + MLT 800 * | 0 | 0 |

The data are representative of three independent experiments.
The cell viability was represented as Average ± S:D.
Table includes data of cell viability (% vs vehicle) as a function of dose (μgrams/ml) showing that CBD, MLT and AKBA induced cytotoxicity in both cell lines, with different potency, according to exemplary embodiments of the present disclosure.
Cell lines were treated with daily administration of different doses of the compounds as indicated on the Table.
Cell viability was evaluated at 72 hours post-treatment, by MTT assay.
Data shown are expressed as mean ± SE of three separate experiments and six wells for each dose.
Synergism was indicated with an *.

### Antitumor Efficacy of Cannabidiol and Melatonin, in a Nude Mice Orthotopic Pancreatic Tumor Model

### Aim of the Study

The objective of this study was to determine the efficacy of the antitumor compounds namely Melatonin (MLT), Cannabidiol (CBD) in a nude mice orthotopic pancreatic tumor model. The effect of the compounds will be tested as a mixture of MLT and CBD together (MLT+CBD), and in combination with Gemcitabine (GEM).

The efficacy of the treatments will be evaluated by measuring in vivo the tumor volume, and therapeutic compounds efficacy.

### Test System

Athymic nude mice were chosen as test system since this specie and strain are widely used in literature as a suitable model for this kind of study.

Species and strain: Athymic Nude-Foxn1nu mice.

Number and sex of animals: 20 females

Weight and age at arrival: 20-25 gr, 5 weeks old.

Supplier: Envigo RMS SARL, Gannat, France.

### Animal Husbandry

Food: VRF1(P)QC pelleted diet produced by SPECIAL DIET SERVICES, Whitam, Essex (UK). The Producer will supply a certificate of analysis for nutrients and contaminants, the level of the latter to be within the limits proposed by EPA-TSCA (44FR: 44053-44093, Jul. 26, 1979). Food will be available “ad libitum”.

Water: tap water from the municipal water supply, filtered through 1.0 and 0.2 μm filters. 3 mL of gentamicin sulfate antibiotic (50 mg/mL) will be added to 1 liter of water to reach a final concentration of 150 mg/L. Water will be available “ad libitum”.

Bedding material: Yelu XYL® HW 300/500 will be supplied by Charles River and certified as being without contaminant in toxic concentrations.

Housing: animals will be housed in type 3 Makrolon® Tecniplast cages during the study and each cage will house a maximum of 5 mice.

Environmental conditions: during the entire period of the study the animals will be maintained in conditioned and at limited access environments. The parameters are set as follows:
mean temperature (range values): 22° C. (20-24° C.)
mean relative humidity (range values): 55% (45-65%)
air changes: 15 to 20 per hour
lighting: controlled by automatic clock to give a daily 12 h photoperiod.

Temperature and relative humidity data will be recorded every 10 seconds by a computerized data base (TrendManager Pro V5, Honeywell); a daily mean value will be calculated, and raw data will be stored at CEIP.

All the procedures involving the animals will be conducted according to the national and international laws on experimental animal (d.l. 4 Mar. 2014, implementation of directive n. 2010/63/UE) and to the approved experimental protocol procedure (Authorization n° 844/2021-PR). No validated non-animal alternatives are known to meet the objectives of the study.

Animals will be identified and numbered within each group by ear sign. Study number, animal number, group, dosage, and date of compound administration will be reported on each cage card.

### Methods

### In Vivo Experimental Design

### Cell Culture

PANC-1 cells were thawed and cultured in DMEM (Dulbecco's Modified Eagle Medium) +2% L-glutamine +10% FBS (Fetal Bovine Serum), Penicillin/Streptomycin and maintain the cell culture in 100 mm Petri dish (Sarstedt, Germany).

### Cell Preparation and Tumor Inoculation

A period of acclimation (at least 5 days) was allowed to mice before tumor inoculation. Before the inoculum, PANC-1 cells were detached with trypsin, counted with trypan blue and for each inoculum, 1×10⁶ cells were suspended in 20 ul of PBS. Mice (n=20) were anesthetized with isoflurane (induction at 4% and maintenance at 2%) and cell solutions (20 μL, 1×10⁶ cells) were orthotopically injected into the tail of the pancreas exploiting the echo-guided procedure. The 20 animals inoculated with tumor cells were divided in 4 groups (Table 6) when tumor size reaches a volume of approximately 20-30 mm³ (tumor size was determined by ultrasound imaging analysis).

TABLE 6
animal treatments;

| | n. of | | | |
|---|---|---|---|---|
| Groups | animals | Treatment | Dose | Adm. route |
| 1 | 5 | Vehicle | | IP |
| | | (saline) | | |
| 2 | 5 | GEM | 50 mg/kg | IP |
| 3 | 5 | MLT + CBD | 400 mg/kg (melatonin) | IP |
| | | | & | |
| | | | 10 mg/kg (cannabidiol) | |
| 4 | 5 | MLT + CBD | 400 mg/kg (melatonin) | IP |
| | | + | & | |
| | | GEM | 10 mg/kg (cannabidiol) + | |
| | | | 50 mg/kg | |
| | | | (Gemcitabine) | |

### Experimental Design

The animals were inoculated with 1×10⁶ PANC-1 cells with VevoLAZR-X system by using echo-guided injection method. All animals were treated every three days for 30 days starting when tumor reached the volume of approximately 30 mm³ (around 10-15 days from the inoculum). From the beginning of the treatments, for the following 4 weeks, ultrasound and photoacoustic imaging were performed to evaluate the development of tumor masses. At the end point mice were sacrificed and macroscopic necroscopy were performed.

Statistical Analysis All data are presented as mean±S.D. For comparison of the statistical differences of more than two groups, oneway ANOVA and Student' unpaired t-test using the Prism GraphPad software (San Diego, CA, USA). A p value of <0.05 was considered statistically significant.

### In Vivo Experiments

### Data Analysis

Tumor volume was analyzed by using Vevo Lab software (Fujifilm Visualsonics).

### Results

We determined whether the combination of MLT 30 CBD alone or with GEM (as positive control) respect to vehicle alone was able to reduce the growth of PANC-1 cells in an orthotopic xenograft nude mouse model (FIG. 1A). Twenty mice were orthotopically injected with PANC-1 cells and randomly assigned to four groups (n=5 per group). Treatment with GEM, MLT+CBD, GEM+MLT+CBD and vehicle control began 19 days after injection and lasted for 30 days. Compared with the control group after 30 days of treatment, the tumor volumes were: Vehicle (630,8)

GEM (111,1), MLT+CBD (291,6), GEM+MLT+CBD (55,3). GEM and MLT+CBD alone showed a high effect, but together GEM+MLT+CBD evidenced the largest reduction on tumor volume (FIG. 1B) and similar trend but with less efficacy was observed for tumor weight (FIG. 1C).

No significant weight loss (FIG. 1D) or signs of delayed toxicity (tissues weight) were observed in liver (FIG. 1E), while a lower effect was observed for heart (FIG. 1F) and brain (FIG. 1G) in mice after treatments. Collectively these in vivo results indicated that PDAC cells treated with the combination of GEM+MLT+CBD achieve significant cell killing not only in vitro but also in vivo. Moreover, considering that CBD and MLT have a low toxicity in human compared with GEM, these data evidenced that the combination of MLT+CBD alone on as integrative therapy with GEM have strong antitumoral effects on PDAC.

### FULL CITATIONS FOR DOCUMENTS REFERRED TO IN THE DESCRIPTION-REFERENCES

1) Siegel, R.L.; Miller, K.D.; Jemal, A. Cancer statistics, 2018. CA Cancer J. Clin. 2018, 68, 7-30.
2) Adamska, A.; Domenichini, A.; Falasca, M. Pancreatic Ductal Adenocarcinoma: Current and Evolving Therapies. Int. J. Mol. Sci. 2017, 18, 1338.
3) Nabissi, M.; Morelli, M.B.; Odani, M.; Amantini, C.; Gentili, S.; Soriani, A.; Cardinali, C.; Leoni, P.; Santoni, G. Cannabinoids synergize with carfilzomib, reducing multiple myeloma cells viability and migration. Oncotarget 2016, 7, 77543-77557.
4) Velasco, G .; Sánchez, C.; Guzmán, M. Anticancer mechanisms of cannabinoids. Curr. Oncol. 2016, 23, S23-S32.
5) Marinelli, O.; Morelli, M.B.; Annibali, D.; Aguzzi, C.; Zeppa, L.; Tuyaerts, S.; Amantini, C.; Amant, F.; Ferretti, B.; Maggi, F.; et al. The Effects of Cannabidiol and Prognostic Role of TRPV2 in Human Endometrial Cancer. Int. J. Mol. Sci. 2020, 21, 5409.
6) Jeong, S.; Yun, H.K.; Jeong, Y.A.; Jo, M.J.; Kang, S.H.; Kim, J.L.; Kim, D.Y.; Park, S.H.; Kim, B.R.; Na, Y.J.; et al. Cannabidiol-induced apoptosis is mediated by activation of Noxa in human colorectal cancer cells. Cancer Lett. 2019, 447, 12-23.
7) Morelli, M.B.; Odani, M.; Alesiani, F.; Discepoli, G.; Liberati, S.; Olivieri, A.; Santoni, M.; Santoni, G.; Leoni, P.; Nabissi, M. The effects of cannabidiol and its synergism with bortezomib in multiple myeloma cell lines. A role for transient receptor potential vanilloid type-2 Int. J. Cancer 2014, 134, 2534-2546.
8) Luongo M, Marinelli O, Zeppa L, Aguzzi C, Morelli MB, Amantini C, Frassineti A, di Costanzo M, Fanelli A, Santoni G, Nabissi M. Cannabidiol and Oxygen-Ozone Combination Induce Cytotoxicity in Human Pancreatic Ductal Adenocarcinoma Cell Lines. Cancers (Basel). 2020 Sep. 27;12(10):2774.
9) Yang Y, Huynh N, Dumesny C, Wang K, He H, Nikfarjam M. Cannabinoids Inhibited Pancreatic Cancer via P-21 Activated Kinase 1 Mediated Pathway. Int J Mol Sci. 2020;21(21):8035.
10) Ferro, R.; Adamska, A.; Lattanzio, R.; Mavrommati, I.; Edling, C.E.; Arifin, S.A.; Fye, C.A.; Sala, G.; Sacchetto, L.; Chiorino, G.; et al. GPR55 signalling promotes proliferation of pancreatic cancer cells and tumour growth in mice, and its inhibition increases effects of gemcitabine. Oncogene 2018, 37, 6368-6382.
11) Tamtaji OR, Mirhosseini N, Reiter RJ, Behnamfar M, Asemi Z. Melatonin and pancreatic cancer: Current knowledge and future perspectives. J Cell Physiol. 2019;234(5):5372-5378.
12) Syrovets T, Buchele B, Krauss C, Laumonnier Y, Simmet T. Acetyl-boswellic acids inhibit lipopolysaccharide-mediated TNF-α induction in monocytes by direct interaction with IκB kinases. J Immunol. 2005; 174:498-506.
13) Takada Y, Ichikawa H, Badmaev V, Aggarwal BB. Acetyl-11-keto-β-boswellic acid potentiates apoptosis, inhibits invasion, and abolishes osteoclastogenesis by suppressing NF-κB and NF-κB regulated gene expression. J Immunol. 2006; 176:3127-40.
14) Liu JJ, Nilsson A, Oredsson S, Badmaev V, Zhao WZ, Duan RD. Boswellic acids trigger apoptosis via a pathway dependent on caspase-8 activation but independent on Fas/Fas ligand interaction in colon cancer HT-29 cells. Carcinogenesis. 2002; 23:2087-93.
15) Lu M, Xia L, Hua H, Jing Y. Acetyl-keto-β-boswellic acid induces apoptosis through a death receptor 5-mediated pathway in prostate cancer cells. Cancer Res. 2008; 68:1180-6.
16) Zhao W, Entschladen F, Liu H, Niggemann B, Fang Q, Zaenker KS, Han R. Boswellic acid acetate induces differentiation and apoptosis in highly metastatic melanoma and fibrosarcoma cells. Cancer Detect Prev. 2003; 27:67-75.
17) Liu JJ, Nilsson A, Oredsson S, Badmaev V, Duan RD. Keto- and acetyl-keto-boswellic acids inhibit proliferation and induce apoptosis in Hep G2 cells via a caspase-8 dependent pathway. Int J Mol Med. 2002; 10:501-5.
18) Hostanska K, Daum G, Saller R. Cytostatic and apoptosis-inducing activity of boswellic acids toward malignant cell lines in vitro. Anticancer Res. 2002; 22:2853-62.
19) Park B, Sung B, Yadav VR, Cho SG, Liu M, Aggarwal BB. Acetyl-11-keto-β-boswellic acid suppresses invasion of pancreatic cancer cells through the downregulation of CXCR4 chemokine receptor expression. Int J Cancer. 2011;129(1):23-33.

What is claimed is:

1. A method of treating a pancreatic cancer, comprising administering a combination of cannabidiol, melatonin, and AKBA to the subject in need thereof.

2. The method of claim 1, wherein the combination of cannabidiol, melatonin, and AKBA is administered in combination with at least one other anticancer treatment.

3. The method of claim 1, wherein the subject is a human.

4. A method of treating a PC in a subject in need thereof, comprising administering an effective amount of at least one compound to the subject.

5. The method of claim 4, wherein the at least one compound comprises cannabidiol, melatonin, AKBA, or combinations thereof.

6. The method of claim 4, wherein the at least one compound cannabidiol, melatonin, AKBA, or combinations thereof.

7. The method of claim 4, wherein the at least one compound is cannabidiol

8. The method of claim 4, wherein the at least one compound is melatonin.

9. The method of claim 4, wherein the at least one compound is a combination of cannabidiol and melatonin.

10. The method of claim 9, wherein the at least one compound is a combination comprising cannabidiol.

11. The method of claim 10, wherein the combination further consists of melatonin.

12. The method of claim 11, wherein the combination further consists of AKBA.

13. The method of claim 1, wherein the amount of CBD, MLT and AKBA administered is up to 1 mg.

14. The method of claim 1, wherein the amount of CBD administered ranges from 1 microgram to 1 mg.

15. The method of claim 1, wherein the amount of MLT administered ranges from 10 micrograms to 1 mg.

16. The method of claim 1, wherein the amount of AKBA administered ranges from 1 microgram to 1 mg.

17. The method of claim 1, wherein the CBD, MLT and AKBA was administered daily for 72 hours.

18. The method of claim 1, wherein the concentration of CBD administered ranges from 1.9 mg/ml to 15.2 mg/ml.

19. A composition for treating a pancreatic cancer, comprising cannabidiol, melatonin, and AKBA.

20. The composition of claim 19, wherein amount of CBD administered ranges from 1 microgram to 1 mg.

21. The composition of claim 19, wherein the amount of MLT administered ranges from 10 micrograms to 1 mg.

22. The composition of claim 19, wherein the amount of AKBA administered ranges from 1 microgram to 1 mg.

23. The composition of claim 19, wherein the CBD, MLT and AKBA produce a synergistic effect to treat pancreatic cancer.

24. The method of claim 1, wherein the CBD, MLT and AKBA produce a synergistic effect to treat pancreatic cancer.

25. The method of claim 1, wherein administration of cannabidiol, melatonin, and AKBA produces a percent viability of less than 10%, thereby effectively killing PC cells.
